(19) United States
(12) Patent Application Publication
(10) Pub. No.: US 2026/0074079 A1
(43) Pub. Date: Mar. 12, 2026

(54) SYSTEMS AND METHODS FOR EXTRACTING INFORMATION FROM AND SCORING A CONSULTATION BETWEEN A HEALTHCARE PROVIDER AND A PATIENT

(71) Applicant: CEDARS-SINAI MEDICAL CENTER, Los Angeles, CA (US)

(72) Inventors: Timothy DASKIVICH, Los Angeles, CA (US); Michael LUU, Bassett, CA (US)

(73) Assignee: CEDARS-SINAI MEDICAL CENTER, Los Angeles, CA (US)

(21) Appl. No.: 19/390,134

(22) Filed: Nov. 14, 2025

Related U.S. Application Data
(63) Continuation-in-part of application No. PCT/US2024/029526, filed on May 15, 2024.
(60) Provisional application No. 63/502,606, filed on May 16, 2023.

(51) Int. Cl. G16H 50/70; G16H 15/00; G16H 80/00

(52) U.S. Cl. CPC G16H 50/70; G16H 15/00; G16H 80/00

(57) ABSTRACT

A method of extracting information from a consultation between a healthcare provider and a patient and subsequently scoring the consultation comprises receiving data associated with a transcript of the consultation between the healthcare provider and the patient. The method further comprises analyzing the data to extract a plurality of word groups, each word group including or more words spoken by the healthcare provider during the consultation. The method further comprises determining whether each respective word group of the plurality of word groups is associated with a predetermined topic. The method may further comprise assigning a score to at least one word group of the plurality of word groups that is determined to be associated with the predetermined topic, the score indicating a level of detail of the at least one word group.

### CROSS-REFERENCE TO RELATED APPLICATIONS

This application is a continuation-in-part of PCT Application No. PCT/US2024/029526, filed May 15, 2024, which claims priority to and the benefit of U.S. Provisional Patent Application No. 63/502,606, filed May 16, 2023, which is hereby incorporated by reference herein in its entirety.

### STATEMENT REGARDING FEDERALLY SPONSORED RESEARCH AND DEVELOPMENT

This invention was made with government support under Grant No. CA230155 awarded by the National Institutes of Health. The government has certain rights in the invention.

### TECHNICAL FIELD

The present invention relates generally to models for extracting information from a consultation between a healthcare provider and a patient and scoring the quality of communication of the consultation, and more specifically, to methods for generating and implementing one or more random forest models for identifying word groups containing such key information related to tradeoffs associated with treatment decision, and using the word groups for scoring the consultation.

### BACKGROUND

Consultations between a healthcare provider and a patient are an opportunity for the healthcare provider to explain to the patient various different aspects of a medical condition that the patient may have, include their life expectancy, the prognosis of the medical condition, side effects of the medical condition and/or treatments for the medical condition, etc. However, it can be difficult for the patient to remember the details of key aspects of the consultation when they are considering treatment options after the consultation. Patients are increasingly requesting that consultations be recorded so that information from the consultation can be revised to enhance understanding and retention of key facts. Providing records can also improve patients' knowledge, satisfaction with treatment, and relationship with their healthcare provider. Patients with cancer are often particularly interested in recording consultations, which may stem from the complexity and gravy of the information discussed, as well as the increased difficulty of retaining such information due to the emotionally evocative nature of cancer. Yet without proper medical knowledge and understanding of key tradeoffs information a treatment decision, it can be difficult to parse through a clinical consultation, which often lasts between 30 and 60 minutes. Thus, new systems and methods are needed for extracting information from consultations, so that such information can be reported back to the patient in a manner that helps improve their decision-making.

In addition, the quality of communication in consultations can vary widely based on how much detail the healthcare provider provides to the patient. For example, a generalized statement about the patient's life expectancy (e.g., “you have a long life expectancy”) can be less helpful than a more detailed, patient-specific statement about the patient's life expectancy (e.g., “based on your health and other physiological characteristics, you have an X % chance of dying after Y years”). However, it can be difficult for a healthcare provider to obtain feedback about their performance during consultations, which could be used to improve their skills and offer future patients more detailed and helpful consultations. Thus, new systems and methods for scoring consultations between healthcare providers and patients are needed.

### SUMMARY

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

According to some implementations of the present disclosure, a method of reviewing a consultation between a healthcare provider and a patient comprises receiving data associated with a transcript of the consultation between the healthcare provider and the patient. The method further comprises analyzing the data to extract a plurality of word groups, each word group including or more words spoken by the healthcare provider during the consultation. The method further comprises determining whether each respective word group of the plurality of word groups is associated with a predetermined topic. The method may further comprise assigning a score to at least one word group of the plurality of word groups that is determined to be associated with the predetermined topic, the score indicating a level of detail of the at least one word group.

According to some implementations of the present disclosure, a method of generating a random forest model to review a consultation between a healthcare provider and a patient comprises receiving data associated with one or more consultations between the healthcare provider and the patient. The method further comprises analyzing the data to extract a plurality of word groups. The method further comprises forming a training dataset from a first portion of the plurality of word groups, the training dataset including a (i) plurality of training word groups and (ii) for each respective training word group, an indication of whether the respective training word group is associated with a predetermined topic. The method further comprises generating a plurality of random forest models based on the plurality of training word groups, each of the plurality of random forest models being configured to determine if each of the plurality of training word groups is associated with the predetermined topic. The method further comprises selecting one of the plurality of random forest models, the selected random forest model having a highest accuracy in determining whether each of the plurality of training word groups is associated with the predetermined topic among all of the plurality of random forest models.

According to some implementations of the present disclosure, a method of reviewing a consultation between a healthcare provider and a patient comprises receiving data associated with the consultation, the data including audio data reproducible as audio of the consultation, video data reproducible as a video of the consultation, or both. The method further comprises extracting a transcript of the consultation from the received data. The method further comprises identifying a plurality of word groups within the transcript, each word group including one or more words spoken by the healthcare provider during the consultation. The method further comprises determining a probability that each respective word group is associated with a predetermined topic based on which of a plurality of predetermined tokens are identified in the respective word group. The method further comprises determining an overall score for the consultation based at least in part on the determined probability for at least one of the plurality of word groups.

According to some implementations of the present disclosure, a method of reviewing a consultation between a healthcare provider and a patient comprises receiving data associated with the consultation, the data including audio data reproducible as audio of the consultation, video data reproducible as a video of the consultation, or both. The method further comprises extracting a transcript of the consultation from the received data. The method further comprises identifying a plurality of word groups within the transcript, each word group including one or more words spoken by the healthcare provider during the consultation. The method further comprises determining whether each respective word group is associated with a predetermined topic based on which of a plurality of predetermined tokens are identified in the respective word group. The method further comprises for each respective word group determined to be associated with the predetermined topic, assigning a score to the respective word group based on the tokens of the plurality of predetermined tokens identified in the respective word group, each of the plurality of predetermined tokens being associated with at least one score level of a plurality of score levels. The method further comprises determining an overall score for the consultation based on the score assigned to each word group determined to be associated with the predetermined topic.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure, and its advantages and drawings, will be better understood from the following description of representative embodiments together with reference to the accompanying drawings. These drawings depict only representative embodiments and are therefore not to be considered as limitations on the scope of the various embodiments or claims.

FIG. 1 is a block diagram of a system for training and/or implementing one or more models for extracting information from a consultation between a healthcare provider and a patient and scoring the consultation, according to aspects of the present disclosure.

FIG. 2 is a flowchart of a method for extracting information from a consultation between a healthcare provider and a patient and scoring the consultation, according to aspects of the present disclosure.

FIG. 3 is a flowchart of a method for generating a model for extracting information from a consultation between a healthcare provider and a patient and scoring the consultation, according to aspects of the present disclosure.

FIG. 4A is an ROC curve for internal validation of a first model for extracting word groups related to urinary incontinence from a consultation between a healthcare provider and a patient, according to aspects of the present disclosure.

FIG. 4B is a linear regression plot showing the relationship between the probability of each word group extracted by the first model being related to urinary incontinence and a manual score given to each word group.

FIG. 4C is an ROC curve for external validation of the first model, according to aspects of the present disclosure.

FIG. 5A is an ROC curve for internal validation of a second model for extracting word groups related to lower urinary tract symptoms from a consultation between a healthcare provider and a patient, according to aspects of the present disclosure.

FIG. 5B is a linear regression plot showing the relationship between the probability of each word group extracted by the second model being related to lower urinary tract symptoms and a manual score given to each word group.

FIG. 5C is an ROC curve for external validation of the second model, according to aspects of the present disclosure.

FIG. 6A is an ROC curve for internal validation of a third model for extracting word groups related to erectile dysfunction from a consultation between a healthcare provider and a patient, according to aspects of the present disclosure.

FIG. 6B is a linear regression plot showing the relationship between the probability of each word group extracted by the third model being related to erectile dysfunction and a manual score given to each word group.

FIG. 6C is an ROC curve for external validation of the third model, according to aspects of the present disclosure.

FIG. 7A is an ROC curve for internal validation of a fourth model for extracting word groups related to life expectancy from a consultation between a healthcare provider and a patient, according to aspects of the present disclosure.

FIG. 7B is a linear regression plot showing the relationship between the probability of each word group extracted by the fourth model being related to life expectancy and a manual score given to each word group.

FIG. 7C is an ROC curve for external validation of the fourth model, according to aspects of the present disclosure.

FIG. 8A is an ROC curve for internal validation of a fifth model for extracting word groups related to cancer prognosis from a consultation between a healthcare provider and a patient, according to aspects of the present disclosure.

FIG. 8B is a linear regression plot showing the relationship between the probability of each word group extracted by the fifth model being related to cancer prognosis and a manual score given to each word group.

FIG. 8C is an ROC curve for external validation of the fifth model, according to aspects of the present disclosure.

FIG. 9A shows, for each of five different topics, the proportion of sentences within AI-generated summaries of consultations using sentences extracted from those consultations using six different probability thresholds, according to aspects of the present disclosure.

FIG. 9B shows, for each of five different topics, the probability of topic concordance within AI-generated summaries of consultations using sentences extracted from those consultations using six different probability thresholds, according to aspects of the present disclosure.

FIG. 9C shows, for each of five different topics, the probability of a quality score greater than or equal to 3 for AI-generated summaries of consultations using sentences extracted from those consultations using six different probability thresholds, according to aspects of the present disclosure.

FIG. 9D shows, for each of five different topics, the quality score of AI-generated summaries of consultations using sentences extracted from those consultations using six different probability thresholds, according to aspects of the present disclosure.

FIG. 10 shows, for each of five different topics, the difference between the quality score of a consultation and the quality score of an AI-generated summary of the consultation using sentences extracted from the consultation using six different probability thresholds, according to aspects of the present disclosure.

FIG. 11 shows, for each of five different topics, the difference between the quality score of a consultation and the quality score of an AI-generated summary of the consultation using sentences extracted from the consultation using six different probability thresholds and stratified by race, according to aspects of the present disclosure.

### DETAILED DESCRIPTION

Disclosed herein are systems and methods for extracting information from clinical consultations between a healthcare provider and a patient. The information can be key information related to tradeoffs associated with treatment decision making (e.g., life expectancy, prognosis, treatment benefits, treatment side effects, etc.). The systems and methods can also be used for scoring the consultation based at least in part on the extracted information. Further disclosed herein are systems and methods for generating one or more models used to extract information from and/or score a consultation between a healthcare provider and a patient. The model analyzes a transcript of the consultation between the healthcare provider and the patient, and extracts words, phrases, sentences, etc. determined to be the most relevant to some predetermined topic. Different models can be trained and utilized for different topics.

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

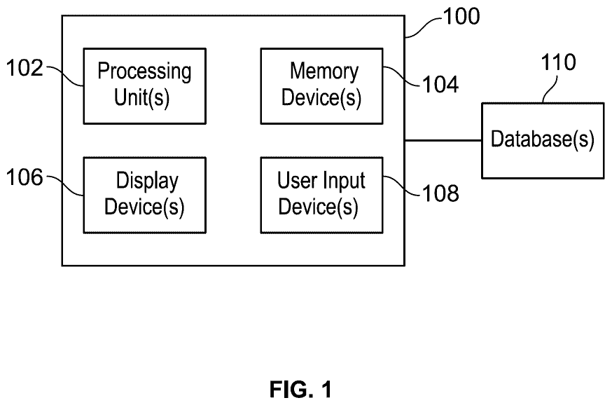

FIG. 1 illustrates a block diagram of system 100 that can be used to implement one or more models for scoring a consultation between a healthcare provider and a patient, and/or to generate one or more models for scoring a consultation between a healthcare provider and a patient. The system 100 includes one or more processing units 102, one or more memory devices 104, one or more displays 106, and one or more user input devices 108. The one or more processing units 102 can generally include a processing unit of any suitable processing device, including general purpose computer systems, microprocessors, digital signal processors, micro-controllers, application specific integrated circuits (ASICs), programmable logic devices (PLDs) field programmable logic devices (FPLDs), programmable gate arrays (PGAs), field programmable gate arrays (FPGAs), mobile devices such as mobile telephones, personal digital assistants (PDAs), or tablet computers, local servers, remote servers, wearable computers, or the like.

The one or more memory devices 104 can generally include any suitable memory device, including solid-state memories, optical media, magnetic media, random access memory (RAM), read only memory (ROM), a floppy disk, a hard disk, a CD ROM, a DVD ROM, flash memory, any other computer readable medium that is read from and/or written to by a magnetic, optical, or other reading and/or writing system, and the like. The one or more display devices 106 can generally include any suitable display device, such as an LCD display, an LED display, an OLED display, a television, a laptop screen, a touch screen, or the like. The one or more user input devices 108 can generally include any suitable user input device, including a keyboard, a mouse, a microphone (for receiving voice input), a touch screen, and the like.

In some implementations, some elements of the system 100 may be combined into a single device. For example, the display device 106 may be a display of a processing device (e.g., a laptop computer or a tablet computer) that also includes the processing unit 102. In another example, a touchscreen can form both the display device 106 and the user input device 108.

The one or more memory devices 104 can store computer-readable instructions that can be executed by the one or more processing units 102 to implement one or more models to score the consultation between the healthcare provider and the patient, and/or to generate one or more models for scoring a consultation between the healthcare provider and the patient.

In some implementations, the system is communicatively coupled to one or more databases 110. The one or more databases 110 can include any data that is needed by the system 100. For example, the databases 110 may store computer-readable instructions that can be executed by the one or more processing units 102 to aid in implementing the one or more models to score the consultation between the healthcare provider and the patient, and/or to aid in generating one or more models for scoring a consultation between the healthcare provider and the patient. In another example, the one or more databases 110 can store data that is used to generate, train, validate, and/or use the one or more models.

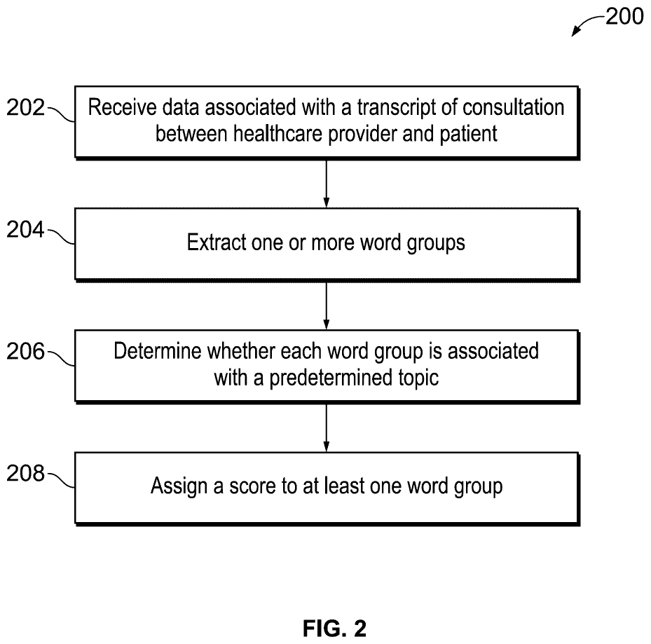

FIG. 2 illustrates a flowchart of a method 200 for scoring a consultation between a healthcare provider and a patient. In general, one or more models can be designed and trained to implement all or part of method 200. Step 202 includes receiving data associated with a transcript of a consultation between a healthcare provider and a patient. The data can include text data, audio data, other types of data, etc. In some implementations, the data includes multiple different types of data. In general, the data will be an electronic transcript of the consultation.

Step 204 includes analyzing the data to extract one or more word groups, where each word group includes one or more words spoken by the healthcare provider during the consultation. In some implementations, each word group contains at least one word. In some implementations, each word group is a single word, a phrase including a plurality of words, a single sentence including a plurality of words, multiple sentences, multiple letters, or any combination thereof. In some implementations, extracting the word groups includes analyzing the data to identify individual sentences, for example by looking for indicators such as periods, capital letters, etc.

Step 206 includes determining whether one or more of the word groups is associated with a predetermined topic. In some implementations, the predetermined topic is a medical condition, a life expectancy following a diagnosis of a medical condition, a result of a treatment of the medical condition (e.g., a reduction in the severity of the medical condition, an eradication of the medical condition, etc.), a side effect of the medical condition, a side effect of a treatment of the medical condition, other topics, or any combination thereof. In some implementations, the medical condition is prostate cancer. In some implementations, the side effect of the medical condition and/or of the treatment includes erectile dysfunction, urinary incontinence, irritative lower urinary tract symptoms, or any combination thereof. In some implementations, the predetermined topic is not a medical condition, but some other topic where analysis of a consultation/consultation would be beneficial.

In some implementations, determining whether each respective word group is associated with the predetermined topic includes determining the probability that the respective word group is associated with the predetermined topic. For example, a probability threshold can be selected, such as 50%, 60%, 70%, etc. The probability that the respective word group is associated with the predetermined topic is determined, and if the probability for the respective word group satisfied the probability threshold (e.g., the probability for the respective word group is equal to or greater than the probability threshold), it is determined that the respective word group is associated with the predetermined topic. If the probability threshold is not satisfied (e.g., the probability for the respective word group is equal to or less than the probability threshold), it is determined that the respective word group is associated with the predetermined topic.

In some implementations, determining whether each respective word group is associated with the predetermined topic includes determining whether the respective word group includes one or more tokens that are associated with the topic. These tokens can be a word, a phrase containing a plurality of words, a word stem, a word root, a word base, etc. For example, if the predetermined topic is cancer prognosis following a cancer diagnosis, the tokens can include “percent,” “cancer,” “likelihood,” chanc,” “caus,” etc., which include both full words and partial words (e.g., “caus” relating to “cause,” “causing,” “caused,” etc.). In general, a token is any letter or group of letters of interest within a given word group.

In some implementations, the determination of whether a respective word group is associated with the predetermined topic is based on a plurality of distinct estimates of whether the respective word group is associated with the predetermined topic. For example, in some implementations the plurality of distinct estimates can be generated, and then the probability that the respective word group is associated with the predetermined topic can be based on how many of the distinct estimates indicate that the respective word group is associated with the predetermined topic.

In some implementations, the plurality of distinct estimates can be generated by a random forest model, which will generally include a plurality of individual decision trees. Each of these decision trees can generate a distinct estimate of whether the respective word group is associated with the predetermined topic or not.

For example, each decision tree can be designed to determine if the respective word group contains one or more tokens. Each decision tree can include a number of nodes, and at each node, the decision tree determines if the word group contains a token associated with that node. If the word group does contain the token, the node branches off in a first direction to a first subsequent node, or to a result (e.g., a yes/no indication of whether the word group is associated with the predetermined topic). If the word group does not contain the token, the node branches off in a second direction to a second subsequent node, or to a result (e.g., a yes/no indication of whether the word group is associated with the predetermined topic).

The decision tree can iterate through the tokens of its nodes, determine whether the respective word group contains each token, and generate an estimate of whether the respective word group is associated with the predetermined topic based on which tokens are contained in the respective word group. Generally, each of the plurality of decision trees of the random forest model will be designed to check the respective word group for subset of tokens that is selected from a larger set of tokens. In some implementations, each decision tree checks the word groups for the same number of tokens, although generally the actual tokens searched for by each decision tree can vary (even if each decision tree searches for the same number of tokens). In other implementations, different decision trees may check the word groups for different numbers of tokens.

Moreover, the tokens that are searched for by the decision trees of the random forest model can be randomly selected from a larger group of possible tokens. For example, in some implementations the group of possible tokens includes every individual word that is found in the plurality of word groups. The actual tokens that each decision tree searches for within a word group can be randomly selected from this set of possible tokens. In this manner, while each decision tree of the random forest model may search for the same number of tokens, the decision trees may not be searching for the same tokens. In general, the decision trees of the random forest model can check the word groups for any suitable number of tokens. Generally, the output of a given decision tree is a binary determination of whether or not the respective word group is associated with the predetermined topic. However, other decision trees can be utilized.

The percentage of the decision trees that conclude that the respective word group is associated with the predetermined topic can be the probability that the respective word group is associated with the predetermined topic. For example, if 70% of the decision trees conclude that a given word group is associated with the predetermined topic and the remaining 30% of the decision trees conclude that the word group is not associated with the predetermined topic (or do not reach a conclusion), then the probability that the word group is associated with the predetermined topic as determined by the random forests model will be 70%. In implementations using a probability threshold, if this 70% determination satisfies the probability threshold, then the outcome of step 206 of the method 200 for this word group is that this word group is associated with the predetermined topic. If this 70% determination does not satisfy the probability threshold, then the outcome of step 206 of the method 200 for this word group is that this word group is not associated with the predetermined topic.

The method optionally includes step 208, which includes assigning a score to one or more of the word groups. The score is separate from the probability of being associated with the topic that is determined in step 206, and can be based on how much detail the word group provides about the predetermined topic. For example, a word group that only mentions the predetermined topic by name (e.g., “a given side effect might occur”) may receive a low score, whereas a word group that provides patient-specific details about the topic (e.g., “based on your age and physiological characteristics, you have a 30% chance of developing a given side effect”) may receive a higher score. In some implementations, a score for each word group is determined. In other implementations, a score is determined only for word groups having at least a threshold probability of being associated with the topic. In further implementations, a score is determined only for the top-n highest-probability word groups. References herein to scoring word groups will be understood to be to the word groups that are being scored, whether that is all of the word groups or only some of the word groups.

In some implementations, a hierarchy of specificity is used to categorize word groups and assign the score. This hierarchy can be used to rank identified word groups that are related to the topic by the amount of information they provide about the topic, with word groups that provide more information be scored higher. In one example, the predetermined topic is life expectancy (e.g., estimated years of life left) following a specific diagnosis, and the hierarchy includes from least specific to most specific: (0) not mentioning the life expectancy, (1) mentioning life expectancy but not quantifying it, (2) a generalization or binary classification of the life expectancy, (3) a rough number of years, (4) a probability of survival at a certain time point, and (5) a specific number of years. In another example, the predetermined topic is the prognosis of a specific disease (e.g., chances of dying from the disease), and the hierarchy includes from least specific to most specific: (0) not mentioning the prognosis, (1) mentioning the prognosis but not quantifying it, (2) a generalization or binary classification of the prognosis, (3) a probability of mortality with no timeline or based only on treatment or not treatment, (4) a probability of mortality at a specific timepoint number with and without treatment, and (5) a probability of mortality at a specific life expectancy for the patient. In a further example, the predetermined topic is a probability of experiencing a side effect from a disease and/or from a treatment of the disease, and the hierarchy includes from least specific to most specific: (0) not mentioning the side effect, (1) mentioning the only by name, (2) a generalization or binary classification of the probability of experiencing the side effect, (3) an average probability of experiencing the side effect with no timeline, (4) an average probability of experiencing the side effect within a specific point in time in the future, and (5) a probability of experiencing the side effect on patient specific characteristics.

In some implementations, specific tokens are associated with different levels of specificity. In these implementations, determining whether each word group is associated with the predetermined topic can further include classifying each respective word group by the level of specificity associated with the predetermined topic, based on the tokens within the respective word group. In general, all of the word groups determined to be associated with the predetermined topic can further be classified into any number of specific classification levels based on the tokens within each word group, where each token can be associated with a specific one (or more) of the classification levels.

In other implementations, the score for each respective word group can be based on the probability (e.g., determined by a random forest model) that the respective word group is associated with the predetermined topic. For example, in the above-referenced example with score levels from 0-5, a word group with a probability less than 50% can be assigned a score level of 0; a word group with a probability between 50% and 50.99% can be assigned a score level of 1; a word group with a probability between 60% and 60.99% can be assigned a score level of 2; a word group with a probability between 70% and 70.99% can be assigned a score level of 3; a word group with a probability between 80% and 80.99% can be assigned a score level of 4; and a word group with a probability between 90% and 100% can be assigned a score level of 5.

In further implementations, a score can be assigned to a consultation as a whole based on the scores of any number of individual word groups. In some examples, the consultation score is based on the score of every word group within the consultation. In other examples, the consultation score is based on the score of a specific set of word groups within the consultation. This set of word groups may include the top-n highest-probability word groups identified by the model. The n variable may be any suitable number, such as 1, 5, 10, 20, etc. In any example, the scores of each word group in the set of word groups used to score the consultation can be combined (e.g., an average of all of the scores, a weighted average of all of the scores, etc.) to provide an overall score for the entire consultation.

Different word groups could be weighted based on a variety of different factors. In some implementations, word groups are weighted based on when they are spoken during the consultation (e.g., their temporal location within the consultation, their location within text representing the speech of the healthcare provider during the consultation, etc.). In these implementations, word groups appearing earlier in the consultation are weighted more heavily than word groups appearing later in the consultation, based on the idea that it is important for healthcare providers to provide high-specificity information to the patient as soon as possible during the consultation. For example, word groups appearing in the first half of the consultation could be weighted more heavily than word groups appearing in the second half of the consultation. In another example, the consultation could be divided into thirds, with word groups in the first third being weighted higher than word groups in the middle third and the last third, and with word groups in the middle third being weighted higher than word groups in the last third.

In some implementations, word groups are weighted based on their length, based on the idea that it is important for healthcare providers to explain concepts to their patents without overly rambling and not in very short sound bites. In these implementations, word groups that are longer than a high threshold length and/or shorter than a low threshold length may be weighted less heavily than word groups that fall within both length thresholds. In implementations where each word group is a sentence, the length thresholds can be a specific number of words, and the length of each word group is the number of words in the sentence.

In other implementations, the word groups are not first individually scored in order to assign a score to the consultation, but instead the consultation as a whole is scored based on the word groups. For example, the score for the consultation could be the average probability of any number of word groups (e.g., all the word groups, the top n highest-probability word groups, etc.). The probabilities of the word groups may in some cases be weighted, such as by word group length, location within the consultation, etc. In another example, the score for the consultation could be the sum of the individual probabilities of each of the word groups used for the scoring (e.g., all the word groups, the top n highest-probability word groups, etc.).

In some implementation, the scoring of the consultation is based on the percentage of word groups within the consultation that have certain probabilities of being associated with the topic. For example, the score for a consultation can be the percentage of word groups that satisfy a predetermined threshold probability of being related to the predetermined topic, out of all of the word groups in the consultation. This percentage could also be weighted by the specific probability, such that out of all the word groups that satisfy the predetermined threshold probability, word groups with a higher probability are counted more than word groups with a lower probability. For example, if the threshold probability is 50%, word groups having a probability between 50% and 50.99% can be counted once; word groups with a probability between 60% and 60.99% can be counted a times, where a is greater than 1; word groups with a probability between 70% and 70.99% can be counted b times, where b is greater than a; word groups with a probability between 80% and 80.99% can be counted c times, where c is greater than b; and word groups with a probability between 90% and 100% can be counted d times, where d is greater than c. In this example, if there are 20 total word groups and 10 have a probability of at least 50%, the unweighted score given to the consultation can be a 0.5 (e.g., 5/10, 50/100, etc.). However, consider the example where the word groups are weighted based on probability levels, and the 10 word groups with at least 50% probability includes 1 word group between 50% and 50.99%, 2 word groups between 60% and 60.99%, 1 word group between 70% and 70.99%, 3 word groups between 80% and 80.99%, and 3 word groups between 90% and 100%. In this example, if the weight given to the 50% to 50.99% is 1 and the weight increments by 0.2 for each probability level, than the score could be calculated as ((1×1)+ (2×1.2)+ (1×1.4)+ (3×1.6)+ (3×1.8))/20=15/20−0.75 (e.g., 7.5/10, 75/100, etc.).

The scoring can be done automatically be the random forest model and/or by a computing device or system that is implementing the random forest model. For example, once the random forest model identifies the word groups and determines the probability that each word group is associated with the predetermined topic, a score can automatically be assigned to each word group (e.g., based on the tokens within the word group, based on the probability, etc.) which is used to score the consultation, or the consultation as a whole can be scored based on the individual word groups. In other implementations, the scoring can be done manually.

In some implementations, the scoring for a consultation done on a topic-by-topic basis. For example, a consultation may have a first score showing the quality of the information communicated about a first topic (e.g., life expectancy) and a second score showing the quality of the information communicated about a second topic (e.g., treatment outcomes). The score for an individual topic can be generated in any suitable manner, including those discussed herein. In other implementations, the scoring for a consultation is done on an overall basis. In these implementations, the consultation score for each topic can be determined and then combined (e.g., a sum of the scores, an average of the scores, a weighted average of the scores, etc.) to determine the overall score for the consultation.

In some implementations, the method 200 may further comprising transmitting certain information to the patient, the healthcare provider, or some other person/entity. For example, the score determined in optional step 208 could be transmitted. Other information that could be transmitted can include the extracted word groups, a subset of the extracted word groups, (e.g., the word groups with the highest probability of being associated with the predetermined topic, etc.), a list of relevant tokens within the word groups (e.g., the tokens found within the word groups having the highest probability of being associated with the predetermined topic, etc.), a summary of the extracted word groups, etc. In some of these implementations, a summary of the consultation for each topic can be generated (for example using machine learning models such as large language models (LLMs)) using only the top n highest-probability word groups identified by the model, which generally results in the summaries being more direct and to the point for the patient. For example, the n highest-probability word groups for a given topic can be input into an LLM, which can be asked to summarize the topic based in the inputted word groups. This can be done for each topic for a given consultation, so that distinct summaries of the consultation for each topic are available for review (e.g., by the patient, by the healthcare provider, etc.).

In some implementations, the various features disclosed herein can be used to implement a method of automated scoring of the consultation. A first step of this method includes receiving data associated with the consultation. The data can include audio data reproducible as audio of the consultation, video data reproducible as video of the consultation, or both.

A second step of the method includes extracting a transcript of the consultation from the received data.

A third step of the method includes identifying a plurality of word groups within the transcript, where each word group includes one or more words spoken by the healthcare provider during the consultation. In some implementations, each word group is a sentence.

A fourth step of the method includes determining the probability that each respective word group is associated with a predetermined topic based on which of a plurality of predetermined tokens are identified in the respective word group. This determination can be based on any of the features disclosed herein. A fifth step of the method includes determining an overall score for the consultation based at least in part on the determined probability for at least one of the word groups.

In some implementations, the score for the consultation is based on a score of one or more of the word groups. For example, the method can include assigning a score to word groups based on the probability of being associated with the predetermined topic of each word group. In some cases, a score is assigned to only a portion of the entire set of word groups in the consultation, such as those having at least a threshold probability of being associated with the topic, or those in the top-n highest probability word groups (e.g., top 5, top 10, top 20, etc.).

In some implementations, the score of any word group that is scored is based at least in part on the tokens that were identified in each of these word groups. Each of the tokens can be associated with one or more score levels of a plurality of score levels, and thus each word group can be assigned to one of the score levels. The assigning of the score levels to the word groups can be done in any suitable fashion. In some implementations, the score level assigned to a word group is the score level of the plurality of score levels having the most tokens within that word group. For example, if a word group contains one token assigned to score level 3, three tokens assigned to score level 2, and one token assigned to score level 1, that word group can be assigned to score level 2. In some implementations, the score level assigned to a word group is the high score level of the tokens within that word group. Referring to the previous example, the high score level of an individual token within the word group is score level 3, and thus the word group is assigned to score level 3. In some implementations, the score level assigned to a word group is the average score level of all of the tokens within the word group, which may be rounded up or down to the nearest score level. Again referring to the previous example, the average score level of the tokens is score level 2, which is the score level assigned to the word group. If the average level falls between two of the distinct score levels, either score level can be assigned to the word group. In further implementations, the score of each word group can be based on the determined probability that the word group is associated with the predetermined topic, as discussed herein.

In some implementations, the overall score for the consultation is the average score among all of the word groups within the consultation determined to be associated with the predetermined topic. In some implementations, the overall score for the consultation is a weighted average score among all the words groups within the consultation determined to be associated with the predetermined topic. In determining the weighted average, each word group can be weighted based on the location of the word groups within the transcript of the consultation, a length of the word group, and/or any other characteristic or combination of characteristics.

In some implementations, a sixth step of the method includes generating a message containing the overall score for the consultation and transmitting the message to the healthcare provider, the patient, or both. In some cases, this message is automatically generated and transmitted in response to the overall score for the consultation being determined. In some cases, the message includes further information, such as a listing of each word group assigned to the highest score level, a listing of every word group determined to be associated with the predetermined topic and its associated score level, a listing of every word group having at least the threshold probability of being associated with the topic, a listing of the top-n highest-probability word groups, and/or any other suitable information.

Thus, the various features disclosed herein can be used to review and analyze the consultation between the healthcare provider and the patient, and to provide near real-time scoring of the consultation, allowing the healthcare provider to easily review their patient communication skills. This also allows the healthcare provider to work on improving their patient communication skills, and to follow up with a specific patient if the consultation with that patient was scored at a low score level. The scoring can be done automatically be the model and/or a computing device or system implementing the model, but may also be done manually (e.g., as part of the healthcare provider's review of the extracted word groups, by a third party, etc.).

In general, a model can be trained to implement some or all of the methods disclosed herein and/or the related features. Instructions for executing the model can be stored in the memory device 104 of system 100, and the processing unit 102 of the system 100 can execute these instructions to cause the model to implement some or all of the method 200. In some implementations, the model is a random forest model that includes a plurality of decision trees and is configured to implement all steps of method 200. In some implementations, the model is a random forest model that includes a plurality of decision trees and is configured to implement only some steps of method 200 (such as steps 202, 204, and 206; steps 204 and 206; only step 206; etc.).

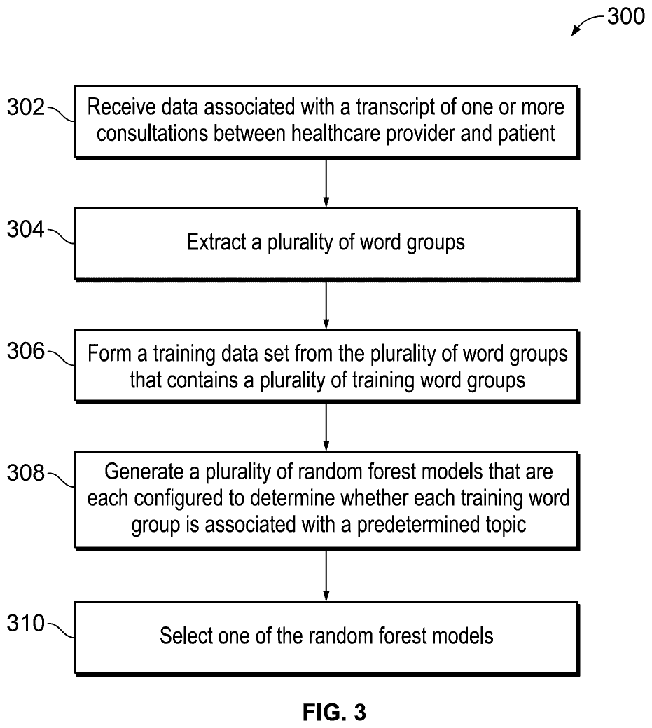

FIG. 3 shows a flowchart of a method 300 for generating a random forest model that can be used to score a consultation between a healthcare provider and a patient. For example, the random forest model that is generated using method 300 can be used to implement all or part of method 200. In general, method 300 can be used to generate a random forest model for a specific predetermined topic, such that method 300 would have to be implemented multiple times if a consultation were to be scored based on multiple different topics.

Step 302 is similar to step 202 and includes receiving data associated with a transcript of one or more consultations between a patient and a healthcare provider. In general, the data does not need to originate from consultations between a single patient and a single healthcare provider, but can instead originate from consultations between a large number of healthcare providers and their patients. Similar to step 202, the data can include text data, audio data, etc.

Step 304 is similar to step 204 and includes extracting a plurality of word groups from the data. In general, each of the word groups includes one or more words spoken by one of the healthcare providers during one of consultations. In some implementations, each word group contains at least one word. In some implementations, each word group is a single word, a phrase including a plurality of words, a single sentence including a plurality of words, multiple sentences, multiple letters, or any combination thereof. In some implementations, extracting the word groups can be done manually.

Step 306 includes forming a training dataset from at least a portion of the plurality of word groups. Generally, the training dataset will include a plurality of training word groups, where the number of words groups in the plurality of training word groups is less than the number of words groups in the plurality of word groups extracted from the data. The training dataset will also include, for each training word group, an indication of whether that training word group is associated with a predetermined topic. Thus, forming the training dataset can include manually determining whether each of the training word groups is associated with the predetermined topic.

Step 308 includes generating a plurality of random forest models based on the plurality of training word groups in the training dataset. Each random forest model is configured to determine whether each of the training word groups is associated with the predetermined topic. In some implementations, each respective random forest model includes a plurality of decision trees, and each decision tree of each random forest model is configured to estimate whether each respective training word group is associated with the predetermined topic. The respective random forest model can then determine the probability that each respective training word group is associated with the predetermined topic based on the estimates about the respective training word group by the decision trees of the respective random forest model.

In some implementations, similar to method 200, each respective random forest model determines the probability that a given training word group is associated with the predetermined topic. This probably can be based on the estimates of the plurality of decision trees of that respective random forest model. For example, the probability determined by the respective random forest model may be the percentage of the decision trees of the respective random forest model that estimate that a given training word group is associated with the predetermined topic.

Similar to method 200, each decision tree will contain a number of nodes that each correspond to a specific token. The decision trees can each iterate through the tokens of their nodes, determine whether the respective word group contains each token, and generate an estimate of whether the respective training word group is associated with the predetermined topic based on which tokens are contained in the respective training word group.

In some implementations, each respective random forest model that is generated at step 308 will include a distinct combination of (i) a number of decision trees contained within the respective random forest model, and (ii) a number of tokens that each decision tree of the respective random forest model searches for (the decision trees of a given random forest model—while each searching for the same number of tokens—may each search for a different set of tokens). For example, the plurality of random forest models can include a first random forest model that includes 100 decision trees that each search the training word groups for 500 tokens, a second random forest model that includes 7,525 decision trees that each search the training word groups for 2,875 tokens, a third random forest model that includes 10,000 decision trees that each search the training word groups for 5,250 tokens, etc. The actual tokens searched for by each decision tree can be selected from all of the individual words contained in the plurality of training word groups. In some implementations, this selection of tokens can be random. In some implementations, the number of decision trees within each random forest model can be between 100 and 10,000. In some implementations, the number of tokens searched for by each decision tree can be between 500 and 10,000.

Finally at step 310, the most accurate random forest model among the plurality of random forest models can be selected as the random forest model to be used for a given predetermined topic. The accuracy of the random forest models can be judged in several ways, including the sensitivity (true positive rate), the specificity (true negative rate), the positive predictive value (percentage of all positives that are true positives), the negative predictive value (percentage of all negatives that are true negatives), the J-statistic (also referred to as the J-index or the Youden's index, defined as the sum of the sensitivity and the specificity minus one), the accuracy (the percentage of all determinations that are true), the area under the receiver operating characteristic curve (ROC curve, formed by plotting sensitivity on the vertical axis and 1-specificity on the horizontal axis), or other metrics.

In some implementations, method 300 can further include forming a validation dataset from at least as portion of the plurality of word groups. The validation dataset includes a plurality of validation word groups, and for each respective validation word group, an indication of whether the respective validation word group is associated with the predetermined topic. Generally, the training dataset and the validation dataset are formed from distinct portions of the plurality of word groups, such that the training word groups are distinct from the validation word groups. In some implementations however, the training dataset and the validation dataset may overlap. In some implementations, the training dataset is a bootstrapped dataset generated from all of the received data.

### EXAMPLES

Below are six examples of the methods 200 and 300 illustrated in FIG. 2 and FIG. 3. In the first five examples, a dataset of multidisciplinary treatment consultations of 42 men undergoing initial treatment consultation for low-risk and/or intermediate-risk prostate cancer was used. The consultations were manually reviewed to extract quotes from the healthcare provider related to life expectancy, cancer prognosis, and side effects (continence, irritative lower urinary tract symptoms, and erectile dysfunction). In the sixth example, sentences related to each of these topics were input into LLMs with an increasing probability threshold to generate topic-specific summaries of each consultation.

### Example 1

In the first example, a plurality of random forest models were generated to identify word groups (e.g., sentences) related to urinary incontinence. There were a total of 17,195 word groups among the 42 transcripts, with 257 word groups related to urinary incontinence and 16,938 word groups not related to urinary incontinence. 75% of the data (12,896 word groups with 195 related to urinary incontinence) was used as the training dataset, and 25% of the data (4,299 word groups with 62 related to urinary incontinence) was used as the internal validation dataset. The plurality of random forest models (each including a certain number of decision trees and a certain number of tokens searched for by each decision trees) is shown in Table 1A below, along with the mean and standard error for both the accuracy of the random forest models (percentage of all determinations that are true) and the area under the ROC curve of the random forest models.

TABLE 1A

| Decision Trees | Tokens | Metric | Mean | Standard Error |
|---|---|---|---|---|
| 100 | 500 | accuracy | 0.9292795 | 0.004290086 |
| 100 | 500 | roc_auc | 0.9764411 | 0.007419972 |
| 2575 | 500 | accuracy | 0.935019 | 0.004318462 |
| 2575 | 500 | roc_auc | 0.9792264 | 0.005639898 |
| 5050 | 500 | accuracy | 0.9352509 | 0.003878871 |
| 5050 | 500 | roc_auc | 0.9792496 | 0.005545654 |
| 7525 | 500 | accuracy | 0.9348633 | 0.00384019 |
| 7525 | 500 | roc_auc | 0.9791151 | 0.005588128 |
| 10000 | 500 | accuracy | 0.9340875 | 0.004212271 |
| 10000 | 500 | roc_auc | 0.9791864 | 0.005641708 |
| 100 | 2875 | accuracy | 0.9437818 | 0.003063926 |
| 100 | 2875 | roc_auc | 0.9796885 | 0.006105745 |
| 2575 | 2875 | accuracy | 0.9434718 | 0.002582989 |
| 2575 | 2875 | roc_auc | 0.9811903 | 0.005526424 |
| 5050 | 2875 | accuracy | 0.9447127 | 0.002914428 |
| 5050 | 2875 | roc_auc | 0.9812269 | 0.005648871 |
| 7525 | 2875 | accuracy | 0.9450227 | 0.002606152 |
| 7525 | 2875 | roc_auc | 0.9812758 | 0.005644507 |
| 10000 | 2875 | accuracy | 0.9442477 | 0.002894228 |
| 10000 | 2875 | roc_auc | 0.9812436 | 0.00558138 |
| 100 | 5250 | accuracy | 0.9399041 | 0.002479341 |
| 100 | 5250 | roc_auc | 0.9791802 | 0.00527076 |
| 2575 | 5250 | accuracy | 0.9458758 | 0.002520908 |
| 2575 | 5250 | roc_auc | 0.9805135 | 0.005683054 |
| 5050 | 5250 | accuracy | 0.9452556 | 0.002788478 |
| 5050 | 5250 | roc_auc | 0.9806044 | 0.005555815 |
| 7525 | 5250 | accuracy | 0.9445577 | 0.002612889 |
| 7525 | 5250 | roc_auc | 0.9805974 | 0.005578146 |
| 10000 | 5250 | accuracy | 0.9446353 | 0.002645697 |
| 10000 | 5250 | roc_auc | 0.9806247 | 0.005507688 |
| 100 | 7625 | accuracy | 0.9399043 | 0.003548718 |
| 100 | 7625 | roc_auc | 0.9785016 | 0.005815013 |
| 2575 | 7625 | accuracy | 0.9456433 | 0.002822221 |
| 2575 | 7625 | roc_auc | 0.9807549 | 0.005512831 |
| 5050 | 7625 | accuracy | 0.9446351 | 0.002648484 |
| 5050 | 7625 | roc_auc | 0.9806061 | 0.005570239 |
| 7525 | 7625 | accuracy | 0.9445577 | 0.002645912 |
| 7525 | 7625 | roc_auc | 0.9806478 | 0.005513929 |
| 10000 | 7625 | accuracy | 0.9448677 | 0.002554395 |
| 10000 | 7625 | roc_auc | 0.9805368 | 0.005590384 |
| 100 | 10000 | accuracy | 0.9404474 | 0.002005493 |
| 100 | 10000 | roc_auc | 0.9792972 | 0.005488965 |
| 2575 | 10000 | accuracy | 0.9439376 | 0.002918678 |
| 2575 | 10000 | roc_auc | 0.980521 | 0.005486097 |
| 5050 | 10000 | accuracy | 0.9442475 | 0.002706296 |
| 5050 | 10000 | roc_auc | 0.9807718 | 0.005431114 |
| 7525 | 10000 | accuracy | 0.9453329 | 0.00259548 |
| 7525 | 10000 | roc_auc | 0.9807678 | 0.005506751 |
| 10000 | 10000 | accuracy | 0.9447126 | 0.002500512 |
| 10000 | 10000 | roc_auc | 0.9806273 | 0.005472055 |

The random forest model selected for the predetermined topic of urinary incontinence is the random forest model with the highest mean area under the ROC curve, which includes 7,525 decision trees and 2,875 tokens. Table 1B below shows the top 100 tokens for this random forest model when validated using the internal validation dataset.

TABLE 1B

| Rank | Variable | Importance |
|---|---|---|
| 1 | tfidf_sentences_urinari | 7.83 |
| 2 | tfidf_sentences_incontin | 7.34 |
| 3 | tfidf_sentences_contin | 6.94 |
| 4 | tfidf_sentences_leakag | 5.79 |
| 5 | tfidf_sentences_pad | 5.71 |
| 6 | tfidf_sentences_sphincter | 5.2 |
| 7 | tfidf_sentences_muscl | 4.93 |
| 8 | tfidf_sentences_urin | 4.01 |
| 9 | tfidf_sentences_surgeri | 3.52 |
| 10 | tfidf_sentences_patient | 2.67 |
| 11 | tfidf_sentences_they'r | 2.46 |
| 12 | tfidf_sentences_leak | 2.04 |
| 13 | tfidf_sentences_bladder | 2.01 |
| 14 | tfidf_sentences_control | 2.01 |
| 15 | tfidf_sentences_flow | 1.77 |
| 16 | tfidf_sentences_need | 1.61 |
| 17 | tfidf_sentences_dai | 1.6 |
| 18 | tfidf_sentences_percent | 1.49 |
| 19 | tfidf_sentences_it' | 1.47 |
| 20 | tfidf_sentences_two | 1.43 |
| 21 | tfidf_sentences_month | 1.43 |
| 22 | tfidf_sentences_exercis | 1.34 |
| 23 | tfidf_sentences_three | 1.29 |
| 24 | tfidf_sentences_thi | 1.24 |
| 25 | tfidf_sentences_diaper | 1.23 |
| 26 | tfidf_sentences_connect | 1.18 |
| 27 | tfidf_sentences_men | 1.13 |
| 28 | tfidf_sentences_new | 1.13 |
| 29 | tfidf_sentences_strengthen | 1.09 |
| 30 | tfidf_sentences_erectil | 1.08 |
| 31 | tfidf_sentences_you'r | 1.07 |
| 32 | tfidf_sentences_remov | 0.93 |
| 33 | tfidf_sentences_gui | 0.92 |
| 34 | tfidf_sentences_now | 0.91 |
| 35 | tfidf_sentences_yeah | 0.91 |
| 36 | tfidf_sentences_ar | 0.9 |
| 37 | tfidf_sentences_bathroom | 0.89 |
| 38 | tfidf_sentences_year | 0.84 |
| 39 | tfidf_sentences_chang | 0.84 |
| 40 | tfidf_sentences_onli | 0.82 |
| 41 | tfidf_sentences_back | 0.8 |
| 42 | tfidf_sentences_bodi | 0.77 |
| 43 | tfidf_sentences_mechan | 0.77 |
| 44 | tfidf_sentences_happen | 0.74 |
| 45 | tfidf_sentences_perman | 0.74 |
| 46 | tfidf_sentences_six | 0.73 |
| 47 | tfidf_sentences_mai | 0.72 |
| 48 | tfidf_sentences_recov | 0.69 |
| 49 | tfidf_sentences_peopl | 0.65 |
| 50 | tfidf_sentences_complet | 0.64 |
| 51 | tfidf_sentences_go | 0.63 |
| 52 | tfidf_sentences_okai | 0.63 |
| 53 | tfidf_sentences_cathet | 0.63 |
| 54 | tfidf_sentences_becom | 0.62 |
| 55 | tfidf_sentences_potenc | 0.57 |
| 56 | tfidf_sentences_right | 0.57 |
| 57 | tfidf_sentences_dysfunct | 0.57 |
| 58 | tfidf_sentences_problem | 0.56 |
| 59 | tfidf_sentences_provid | 0.56 |
| 60 | tfidf_sentences_like | 0.56 |
| 61 | tfidf_sentences_thei | 0.55 |
| 62 | tfidf_sentences_never | 0.55 |
| 63 | tfidf_sentences_last | 0.55 |
| 64 | tfidf_sentences_can | 0.55 |
| 65 | tfidf_sentences_ha | 0.53 |
| 66 | tfidf_sentences_becaus | 0.53 |
| 67 | tfidf_sentences_wai | 0.52 |
| 68 | tfidf_sentences_function | 0.52 |
| 69 | tfidf_sentences_everybodi | 0.52 |
| 70 | tfidf_sentences_sexual | 0.51 |
| 71 | tfidf_sentences_that' | 0.51 |
| 72 | tfidf_sentences_make | 0.51 |
| 73 | tfidf_sentences_issu | 0.51 |
| 74 | tfidf_sentences_enough | 0.5 |
| 75 | tfidf_sentences_first | 0.49 |
| 76 | tfidf_sentences_left | 0.49 |
| 77 | tfidf_sentences_weak | 0.49 |
| 78 | tfidf_sentences_sit | 0.48 |
| 79 | tfidf_sentences_occur | 0.47 |
| 80 | tfidf_sentences_regain | 0.47 |
| 81 | tfidf_sentences_likelihood | 0.45 |
| 82 | tfidf_sentences_come | 0.44 |
| 83 | tfidf_sentences_kegel | 0.43 |
| 84 | tfidf_sentences_wear | 0.43 |
| 85 | tfidf_sentences_prostat | 0.43 |
| 86 | tfidf_sentences_strong | 0.43 |
| 87 | tfidf_sentences_get | 0.43 |
| 88 | tfidf_sentences_squeez | 0.43 |
| 89 | tfidf_sentences_sneez | 0.42 |
| 90 | tfidf_sentences_cough | 0.42 |
| 91 | tfidf_sentences_ten | 0.42 |
| 92 | tfidf_sentences_reli | 0.41 |
| 93 | tfidf_sentences_around | 0.41 |
| 94 | tfidf_sentences_disrupt | 0.41 |
| 95 | tfidf_sentences_tell | 0.41 |
| 96 | tfidf_sentences_period | 0.39 |
| 97 | tfidf_sentences_correct | 0.39 |
| 98 | tfidf_sentences_less | 0.39 |
| 99 | tfidf_sentences_beyond | 0.39 |
| 100 | tfidf_sentences_health | 0.38 |

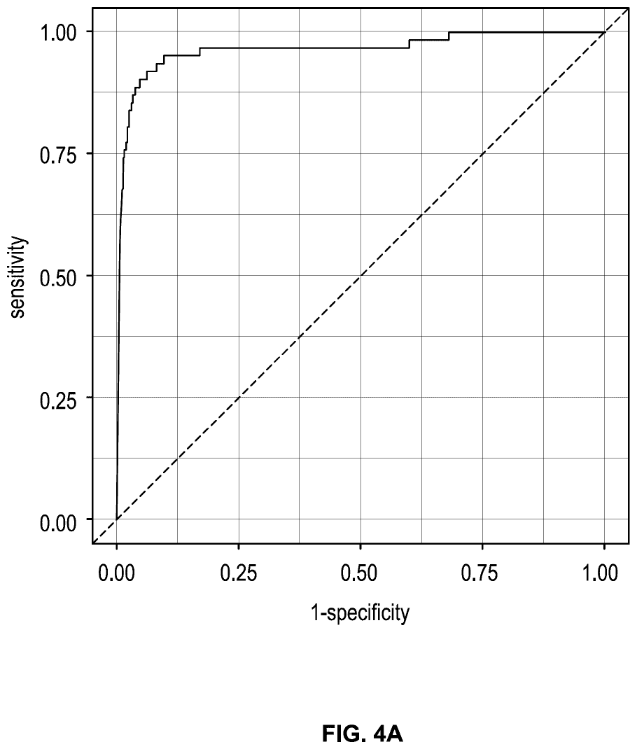

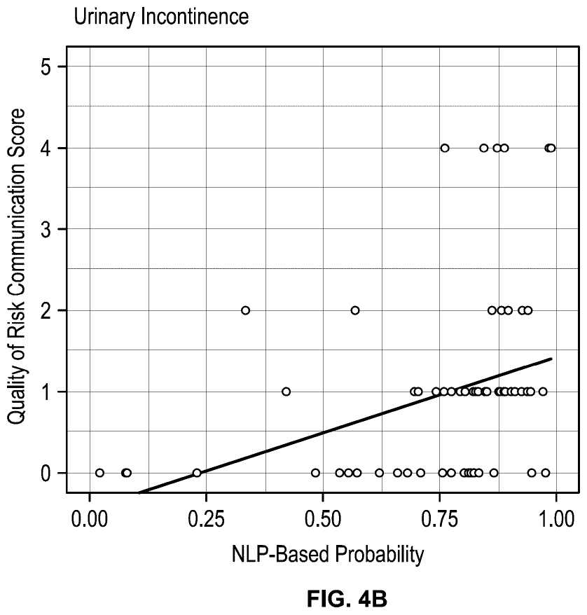

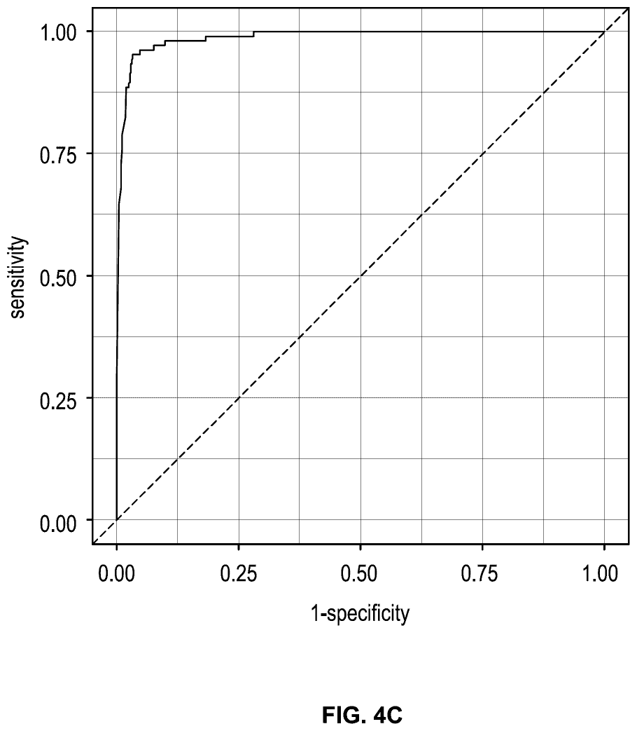

FIG. 4A illustrates the ROC curve for the determination of word groups related to urinary incontinence, plotted against a reference line indicating an equal rate of true positives and false positives. The area under this ROC curve is 0.9648. Table 1C below shows the value of various different statistical parameters of the selected random forest model for different probability thresholds. As the probability threshold increases (e.g., as the threshold for what is expected to be a determination that a given word group is associated with the predetermined topic becomes more restrictive), the sensitivity decreases while the specificity increases. Conversely, as the threshold probability decreases (e.g., as the threshold for what is expected to be a determination that a given word group is associated with the predetermined topic more inclusive), the sensitivity increases while the specificity decreases. The threshold probability that maximizes the sensitivity and specificity by the J-index is 0.5.

TABLE 1C

| Threshold | TN | FP | FN | TP | accuracy | sens | spec | ppv | npv | j_index |
|---|---|---|---|---|---|---|---|---|---|---|
| 0.5 | 3916 | 321 | 4 | 58 | 0.924 | 0.935 | 0.924 | 0.153 | 0.999 | 0.860 |
| 0.6 | 4093 | 144 | 7 | 55 | 0.965 | 0.887 | 0.966 | 0.276 | 0.998 | 0.853 |
| 0.7 | 4180 | 57 | 17 | 45 | 0.983 | 0.726 | 0.987 | 0.441 | 0.996 | 0.712 |
| 0.8 | 4218 | 19 | 31 | 31 | 0.988 | 0.500 | 0.996 | 0.620 | 0.993 | 0.496 |
| 0.9 | 4234 | 3 | 53 | 9 | 0.987 | 0.145 | 0.999 | 0.750 | 0.988 | 0.144 |

However, the selection of the ideal probability threshold for the random forest model would also be informed by the purpose of the information. If the intent is to provide a list of the highest yield information but least inclusive information on urinary incontinence (e.g., containing few word groups that are irrelevant to urinary incontinence at the cost of missing all the word groups having to do with urinary incontinence), then a high probability threshold would be selected. If the intent is to provide a list of the most inclusive but less precise information on urinary incontinence (e.g., containing some word groups that may have less to do with urinary incontinence but capturing more word groups relevant to urinary incontinence), then a low probability threshold would be selected.

For each of the 42 consultations, the top ten word groups (e.g., sentences or phrases spoken by the healthcare provider) by probability of being related to urinary incontinence were used to predict the overall quality of risk communication across the entire consultation using an a priori defined hierarchy for quality assessment. This hierarchy designates quality across a spectrum of increasing granularity of communication: (0) not mentioned, (1) name only (without risk quantification), (2) generalization (“high”/“low”), (3) average percent incidence without timepoint, (4) average percent incidence with timepoint, (5) precision estimate accounting for patient-level characteristics. For each consultation, a reviewer manually analyzed the entire consultation and gave the consultation a score within the hierarchy, where the score generally indicated the highest-scoring word group of all word groups within that consultation. Similarly, a reviewer manually analyzed the top ten word groups identified by selected random forest model and gave the consultation a score within the hierarchy, where the score indicated the highest-scoring word group of the top ten highest-probability word groups. Table 1D below shows the comparison between the highest-scoring word group resulting from the manual review of the entire consultation (“Truth”) and the highest-scoring word group resulting from the manual review of the top ten highest-probability word groups identified by the selected random forest model (“Prediction”). Each cell indicates the number of consultations where the highest-scoring word group was the specific score indicated by the column and row headers of that cell. For example, the cell intersecting the score of 2 for both the Truth axis and the Prediction axis indicates that there were 2 consultations where the highest-scoring word group within the entire consultation was a 2, and the highest-scoring word group within the top ten high-probability word groups identified by the model was also a 2. The 1 in the cell above indicates there was one consultation where the highest-scoring word group out of the entire consultation was a 2, and the highest-scoring word group out of the top ten highest-probability word groups identified by the model was a 1. The ability of the selected random forest model to identify the overall quality of risk communication for urinary incontinence as measured by manual review across the entire consultation showed an 86% accuracy, an 86% sensitivity, a 97% specificity, an 82% positive predictive value, and a 96% negative predictive value.

TABLE 1D

Truth

| | | 0 | 1 | 2 | 3 | 4 | 5 |
|---|---|---|---|---|---|---|---|
| Prediction | 0 | 12 | 0 | 1 | 0 | 0 | 0 |
| | 1 | 0 | 6 | 1 | 0 | 1 | 0 |
| | 2 | 0 | 0 | 2 | 0 | 3 | 0 |
| | 3 | 0 | 0 | 0 | 0 | 0 | 0 |
| | 4 | 0 | 0 | 0 | 0 | 15 | 0 |
| | 5 | 0 | 0 | 0 | 0 | 0 | 1 |

FIG. 4B shows a linear regression plot of the scores given to various word groups for the “urinary incontinence” topic based on manual review, versus the probability that each respective word group is associated with urinary incontinence, with an overlaid trend line. As shown by the plot, the probability of being associated with urinary incontinence is well-correlated with the score given to the word group based on the manual review, indicating that the probability assigned by the model can be used to reliably score a consultation (e.g., grade the quality level of the consultation) with respect to urinary incontinence.

Once the random forest model was selected to identify word groups related to urinary incontinence, it was externally validated using a sample of 13 transcripts containing a total of 4,658 sentences. 105 of the sentences were related to urinary incontinence, and 4,553 sentences were not related to urinary incontinence. FIG. 4C illustrates the ROC curve for this validation, with an area under the curve of 0.987. The model's sensitivity and specificity were calculated for the prediction of word groups related to urinary incontinence at the probability threshold having the highest J-index (0.5) that was identified during training. The model's performance with the external validation dataset using this probability threshold is shown in Table 1E.

TABLE 1E

| | | | | | | | | Balanced |
|---|---|---|---|---|---|---|---|---|
| Threshold | TN | FP | FN | TP | accuracy | sens | spec | Accuracy |
| 0.5 | 4205 | 348 | 4 | 101 | 0.924 | 0.962 | 0.924 | 0.943 |

For each consultation in the external validation dataset, the top ten word groups (e.g., sentences or phrases spoken by the healthcare provider) by probability of being related to urinary incontinence were used to predict the overall quality of risk communication across the entire consultation, using the same hierarchy described above. Table 1F below shows the comparison between the highest-scoring sentence resulting from the manual review of all word groups in the entire consultation and the highest-scoring sentence resulting from the manual review of the top ten highest-probability word groups identified by the selected random forest model. Each cell indicates the number of consultations where the highest-scoring word group was the specific score indicated by the column and row headers of that cell. The ability of the selected random forest model to identify the overall quality of risk communication for urinary incontinence as measured by manual review across the entire consultation showed a 100% accuracy, a 100% sensitivity, and a 100% specificity.

TABLE 1F

Truth

| | | 0 | 1 | 2 | 3 | 4 | 5 |
|---|---|---|---|---|---|---|---|
| Prediction | 0 | 0 | 0 | 0 | 0 | 0 | 0 |
| | 1 | 0 | 0 | 0 | 0 | 0 | 0 |
| | 2 | 0 | 0 | 3 | 0 | 0 | 0 |
| | 3 | 0 | 0 | 0 | 1 | 0 | 0 |
| | 4 | 0 | 0 | 0 | 0 | 9 | 0 |
| | 5 | 0 | 0 | 0 | 0 | 0 | 0 |

### Example 2

In the second example, a plurality of random forest models were generated to identify word groups (e.g., sentences) related to irritative lower urinary tract symptoms. There were a total of 17,195 word groups among the 42 transcripts, with 78 word groups related to irritative lower urinary tract symptoms and 17,117 word groups not related to irritative lower urinary tract symptoms. 75% of the data (12,896 word groups with 57 related to irritative lower urinary tract symptoms) was used as the training dataset, and 25% of the data (4,299 word groups with 21 related to irritative lower urinary tract symptoms) was used as the internal validation dataset. The plurality of random forest models (each including a certain number of decision trees and a certain number of tokens searched for by each decision trees) is shown in Table 2A below, along with the mean and standard error for both the accuracy of the random forest models (percentage of all determinations that are true) and the area under the ROC curve of the random forest models.

TABLE 2A

| Decision Trees | Tokens | Metric | Mean | Standard Error |
|---|---|---|---|---|
| 100 | 500 | accuracy | 0.95906 | 0.00333 |
| 100 | 500 | roc_auc | 0.97761 | 0.00486 |
| 2575 | 500 | accuracy | 0.96069 | 0.00288 |
| 2575 | 500 | roc_auc | 0.97975 | 0.00501 |
| 5050 | 500 | accuracy | 0.96084 | 0.0029 |
| 5050 | 500 | roc_auc | 0.98022 | 0.00484 |
| 7525 | 500 | accuracy | 0.96107 | 0.00302 |
| 7525 | 500 | roc_auc | 0.97997 | 0.0051 |
| 10000 | 500 | accuracy | 0.96038 | 0.00313 |
| 10000 | 500 | roc_auc | 0.98005 | 0.00491 |
| 100 | 2875 | accuracy | 0.96898 | 0.0034 |
| 100 | 2875 | roc_auc | 0.982 | 0.00475 |
| 2575 | 2875 | accuracy | 0.97364 | 0.00246 |
| 2575 | 2875 | roc_auc | 0.98314 | 0.00432 |
| 5050 | 2875 | accuracy | 0.97271 | 0.00265 |
| 5050 | 2875 | roc_auc | 0.9839 | 0.00384 |
| 7525 | 2875 | accuracy | 0.97317 | 0.00262 |
| 7525 | 2875 | roc_auc | 0.98334 | 0.00405 |
| 10000 | 2875 | accuracy | 0.97286 | 0.00267 |
| 10000 | 2875 | roc_auc | 0.98423 | 0.00377 |
| 100 | 5250 | accuracy | 0.96689 | 0.00344 |
| 100 | 5250 | roc_auc | 0.9768 | 0.0065 |
| 2575 | 5250 | accuracy | 0.97263 | 0.00272 |
| 2575 | 5250 | roc_auc | 0.98325 | 0.00398 |
| 5050 | 5250 | accuracy | 0.97302 | 0.00261 |
| 5050 | 5250 | roc_auc | 0.98377 | 0.00395 |
| 7525 | 5250 | accuracy | 0.97255 | 0.00275 |
| 7525 | 5250 | roc_auc | 0.98353 | 0.00415 |
| 10000 | 5250 | accuracy | 0.97271 | 0.00274 |
| 10000 | 5250 | roc_auc | 0.98334 | 0.00404 |
| 100 | 7625 | accuracy | 0.97162 | 0.00407 |
| 100 | 7625 | roc_auc | 0.97741 | 0.00562 |
| 2575 | 7625 | accuracy | 0.97193 | 0.00293 |
| 2575 | 7625 | roc_auc | 0.98221 | 0.00438 |
| 5050 | 7625 | accuracy | 0.97325 | 0.00269 |
| 5050 | 7625 | roc_auc | 0.98346 | 0.00403 |
| 7525 | 7625 | accuracy | 0.97317 | 0.00256 |
| 7525 | 7625 | roc_auc | 0.98431 | 0.00359 |
| 10000 | 7625 | accuracy | 0.97317 | 0.00257 |
| 10000 | 7625 | roc_auc | 0.98311 | 0.00424 |
| 100 | 10000 | accuracy | 0.97115 | 0.00369 |
| 100 | 10000 | roc_auc | 0.97696 | 0.00704 |
| 2575 | 10000 | accuracy | 0.97286 | 0.00268 |
| 2575 | 10000 | roc_auc | 0.98378 | 0.0038 |
| 5050 | 10000 | accuracy | 0.97325 | 0.00258 |
| 5050 | 10000 | roc_auc | 0.98341 | 0.00411 |
| 7525 | 10000 | accuracy | 0.97278 | 0.0027 |
| 7525 | 10000 | roc_auc | 0.98385 | 0.0041 |
| 10000 | 10000 | accuracy | 0.97263 | 0.00276 |
| 10000 | 10000 | roc_auc | 0.98335 | 0.00419 |

The random forest model selected for the predetermined topic of irritative lower urinary tract symptoms is the random forest model with the highest mean area under the ROC curve, which includes 7,525 decision trees and 7,625 tokens. Table 2B below shows the top 100 tokens for this random forest model when validated using the internal validation dataset.

TABLE 2B

| Rank | Variable | Importance |
|---|---|---|
| 1 | tfidf_sentences_urinari | 2.94 |
| 2 | tfidf_sentences_symptom | 1.59 |
| 3 | tfidf_sentences_radiat | 1.31 |
| 4 | tfidf_sentences_urin | 1.27 |
| 5 | tfidf_sentences_bladder | 1.14 |
| 6 | tfidf_sentences_irrit | 1.1 |
| 7 | tfidf_sentences_urgenc | 0.97 |
| 8 | tfidf_sentences_effect | 0.93 |
| 9 | tfidf_sentences_side | 0.79 |
| 10 | tfidf_sentences_frequenc | 0.76 |
| 11 | tfidf_sentences_get | 0.73 |
| 12 | tfidf_sentences_night | 0.67 |
| 13 | tfidf_sentences_wors | 0.56 |
| 14 | tfidf_sentences_term | 0.52 |
| 15 | tfidf_sentences_wake | 0.51 |
| 16 | tfidf_sentences_yeah | 0.44 |
| 17 | tfidf_sentences_go | 0.43 |
| 18 | tfidf_sentences_caus | 0.39 |
| 19 | tfidf_sentences_frequent | 0.31 |
| 20 | tfidf_sentences_cancer | 0.26 |
| 21 | tfidf_sentences_like | 0.25 |
| 22 | tfidf_sentences_feel | 0.21 |
| 23 | tfidf_sentences_just | 0.2 |
| 24 | tfidf_sentences_also | 0.2 |
| 25 | tfidf_sentences_that' | 0.2 |
| 26 | tfidf_sentences_peopl | 0.2 |
| 27 | tfidf_sentences_overact | 0.19 |
| 28 | tfidf_sentences_beam | 0.19 |
| 29 | tfidf_sentences_leakag | 0.18 |
| 30 | tfidf_sentences_think | 0.18 |
| 31 | tfidf_sentences_prostat | 0.17 |
| 32 | tfidf_sentences_mai | 0.17 |
| 33 | tfidf_sentences_you'r | 0.17 |
| 34 | tfidf_sentences_erectil | 0.16 |
| 35 | tfidf_sentences_make | 0.16 |
| 36 | tfidf_sentences_function | 0.15 |
| 37 | tfidf_sentences_need | 0.15 |
| 38 | tfidf_sentences_treatment | 0.15 |
| 39 | tfidf_sentences_treat | 0.15 |
| 40 | tfidf_sentences_want | 0.14 |
| 41 | tfidf_sentences_know | 0.14 |
| 42 | tfidf_sentences_bowel | 0.14 |
| 43 | tfidf_sentences_risk | 0.14 |
| 44 | tfidf_sentences_lot | 0.14 |
| 45 | tfidf_sentences_short | 0.14 |
| 46 | tfidf_sentences_ar | 0.13 |
| 47 | tfidf_sentences_can | 0.13 |
| 48 | tfidf_sentences_expect | 0.13 |
| 49 | tfidf_sentences_pee | 0.13 |
| 50 | tfidf_sentences_men | 0.13 |
| 51 | tfidf_sentences_issu | 0.13 |
| 52 | tfidf_sentences_okai | 0.12 |
| 53 | tfidf_sentences_bad | 0.12 |
| 54 | tfidf_sentences_it' | 0.12 |
| 55 | tfidf_sentences_hit | 0.12 |
| 56 | tfidf_sentences_pretti | 0.12 |
| 57 | tfidf_sentences_kind | 0.12 |
| 58 | tfidf_sentences_stool | 0.12 |
| 59 | tfidf_sentences_surgeri | 0.11 |
| 60 | tfidf_sentences_base | 0.11 |
| 61 | tfidf_sentences_help | 0.11 |
| 62 | tfidf_sentences_time | 0.11 |
| 63 | tfidf_sentences_loos | 0.1 |
| 64 | tfidf_sentences_might | 0.1 |
| 65 | tfidf_sentences_what' | 0.1 |
| 66 | tfidf_sentences_peak | 0.1 |
| 67 | tfidf_sentences_dr | 0.1 |
| 68 | tfidf_sentences_urethra | 0.1 |
| 69 | tfidf_sentences_come | 0.1 |
| 70 | tfidf_sentences_import | 0.1 |
| 71 | tfidf_sentences_you'v | 0.09 |
| 72 | tfidf_sentences_thei | 0.09 |
| 73 | tfidf_sentences_sbrt | 0.09 |
| 74 | tfidf_sentences_well | 0.09 |
| 75 | tfidf_sentences_worsen | 0.09 |
| 76 | tfidf_sentences_put | 0.09 |
| 77 | tfidf_sentences_hi | 0.09 |
| 78 | tfidf_sentences_thi | 0.09 |
| 79 | tfidf_sentences_thing | 0.09 |
| 80 | tfidf_sentences_littl | 0.08 |
| 81 | tfidf_sentences_becaus | 0.08 |
| 82 | tfidf_sentences_right | 0.08 |
| 83 | tfidf_sentences_baselin | 0.08 |
| 84 | tfidf_sentences_how' | 0.08 |
| 85 | tfidf_sentences_onc | 0.08 |
| 86 | tfidf_sentences_now | 0.08 |
| 87 | tfidf_sentences_gui | 0.08 |
| 88 | tfidf_sentences_week | 0.08 |
| 89 | tfidf_sentences_diarrhea | 0.08 |
| 90 | tfidf_sentences_talk | 0.08 |
| 91 | tfidf_sentences_movement | 0.08 |
| 92 | tfidf_sentences_incontin | 0.08 |
| 93 | tfidf_sentences_number | 0.08 |
| 94 | tfidf_sentences_difficult | 0.08 |
| 95 | tfidf_sentences_two | 0.07 |
| 96 | tfidf_sentences_long | 0.07 |
| 97 | tfidf_sentences_better | 0.07 |
| 98 | tfidf_sentences_they'r | 0.07 |
| 99 | tfidf_sentences_becom | 0.07 |
| 100 | tfidf_sentences_area | 0.07 |

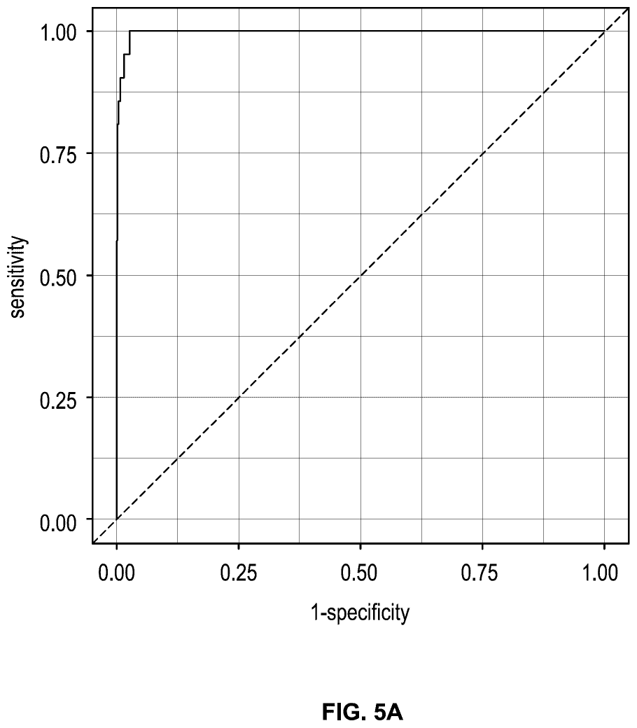

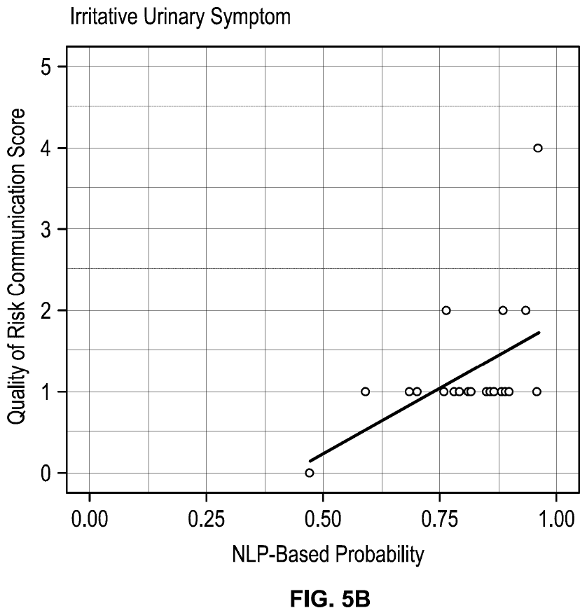

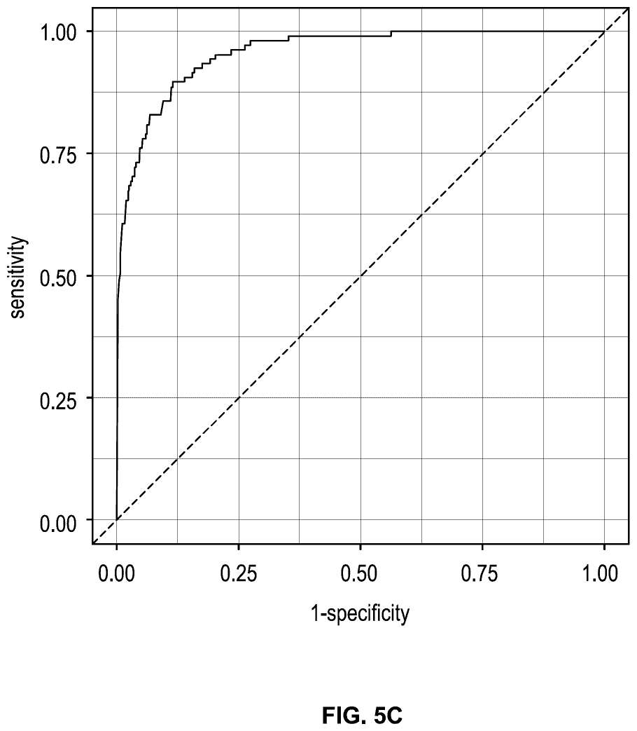

FIG. 5A illustrates the ROC curve for the determination of word groups related to irritative lower urinary tract symptoms, plotted against a reference line indicating an equal rate of true positives and false positives. The area under this ROC curve is 0.9968. Table 2C below shows the value of various different statistical parameters of the selected random forest model for different probability thresholds. As the probability threshold increases (e.g., as the threshold for what is expected to be a determination that a given word group is associated with the predetermined topic becomes more restrictive), the sensitivity decreases while the specificity increases. Conversely, as the threshold probability decreases (e.g., as the threshold for what is expected to be a determination that a given word group is associated with the predetermined topic more inclusive), the sensitivity increases while the specificity decreases. The threshold probability that maximizes the sensitivity and specificity by the J-index is 0.5.

TABLE 2C

| Threshold | TN | FP | FN | TP | accuracy | sens | spec | ppv | npv | j_index |
|---|---|---|---|---|---|---|---|---|---|---|
| 0.5 | 4161 | 117 | 1 | 20 | 0.973 | 0.952 | 0.973 | 0.146 | 1.000 | 0.925 |
| 0.6 | 4250 | 28 | 3 | 18 | 0.993 | 0.857 | 0.993 | 0.391 | 0.999 | 0.851 |
| 0.7 | 4272 | 6 | 7 | 14 | 0.997 | 0.667 | 0.999 | 0.700 | 0.998 | 0.665 |
| 0.8 | 4277 | 1 | 14 | 7 | 0.997 | 0.333 | 1.000 | 0.875 | 0.997 | 0.333 |
| 0.9 | 4278 | 0 | 20 | 1 | 0.995 | 0.048 | 1.000 | 1.000 | 0.995 | 0.048 |

However, the selection of the ideal probability threshold for the random forest model would also be informed by the purpose of the information. If the intent is to provide a list of the highest yield information but least inclusive information on irritative lower urinary tract symptoms (e.g., containing few word groups that are irrelevant to irritative lower urinary tract symptoms at the cost of missing all the word groups having to do with irritative lower urinary tract symptoms), then a high probability threshold would be selected. If the intent is to provide a list of the most inclusive but less precise information on irritative lower urinary tract symptoms (e.g., containing some word groups that may have less to do with irritative lower urinary tract symptoms but capturing more word groups relevant to irritative lower urinary tract symptoms), then a low probability threshold would be selected.

For each of the 42 consultations, the top ten word groups by probability of being related to irritative lower urinary tract symptoms were used to predict the overall quality of risk communication across the entire consultation using an a priori defined hierarchy for quality assessment. This hierarchy designates quality across a spectrum of increasing granularity of communication: (0) not mentioned, (1) name only (without risk quantification), (2) generalization (“high”/“low”), (3) average percent incidence without timepoint, (4) average percent incidence with timepoint, (5) precision estimate accounting for patient-level characteristics. For each consultation, a reviewer manually analyzed the entire consultation and gave the consultation a score within the hierarchy, where the score generally indicated the highest-scoring word group of all word groups within that consultation. Similarly, a reviewer manually analyzed the top ten word groups identified by selected random forest model and gave the consultation a score within the hierarchy, where the score indicated the highest-scoring word group of the top ten highest-probability word groups. Table 2D below shows the comparison between the highest-scoring word group resulting from the manual review of the entire consultation and the highest-scoring word group resulting from the manual review of the top ten highest-probability word groups identified by the selected random forest model. Each cell indicates the number of consultations with the specific scores indicated by the column and row headers of that cell. The ability of the selected random forest model to identify the overall quality of risk communication for irritative lower urinary tract symptoms as measured by manual review across the entire consultation showed an 88% accuracy, an 82% sensitivity, a 96% specificity, an 86% positive predictive value, and a 96% negative predictive value.

TABLE 2D

Truth

| | | 0 | 1 | 2 | 3 | 4 | 5 |
|---|---|---|---|---|---|---|---|
| Prediction | 0 | 19 | 0 | 0 | 0 | 1 | 0 |
| | 1 | 0 | 12 | 0 | 0 | 1 | 0 |
| | 2 | 0 | 3 | 4 | 0 | 0 | 0 |
| | 3 | 0 | 0 | 0 | 0 | 0 | 0 |
| | 4 | 0 | 0 | 0 | 0 | 2 | 0 |
| | 5 | 0 | 0 | 0 | 0 | 0 | 0 |

FIG. 5B shows a linear regression plot of the scores given to various word groups for the “irritative lower urinary tract symptoms” topic based on manual review, versus the probability that each respective word group is associated with irritative lower urinary tract symptoms, with an overlaid trend line. As shown by the plot, the probability of being associated with irritative lower urinary tract symptoms is well-correlated with the score given to the word group based on the manual review, indicating that the probability assigned by the model can be used to reliably score a consultation (e.g., grade the quality level of the consultation) with respect to irritative lower urinary tract symptoms.

Once the random forest model was selected to identify word groups related to irritative lower urinary tract symptoms, it was externally validated using a sample of 13 transcripts containing a total of 4,658 sentences. 104 of the sentences were related to irritative lower urinary tract symptoms, and 4,554 sentences were not related to irritative lower urinary tract symptoms. FIG. 5C illustrates the ROC curve for this validation, with an area under the curve of 0.955. The model's sensitivity and specificity were calculated for the prediction of word groups related to urinary incontinence at the probability threshold having the highest J-index (0.5) that was identified during training. The model's performance with the external validation dataset using this probability threshold is shown in Table 2E.

TABLE 2E

| | | | | | | | | Balanced |
|---|---|---|---|---|---|---|---|---|
| Threshold | TN | FP | EN | TP | accuracy | sens | spec | Accuracy |
| 0.5 | 4211 | 343 | 18 | 86 | 0.922 | 0.827 | 0.925 | 0.876 |

For each consultation in the external validation dataset, the top ten word groups (e.g., sentences or phrases spoken by the healthcare provider) by probability of being related to irritative lower urinary tract symptoms were used to predict the overall quality of risk communication across the entire consultation, using the same hierarchy described above. Table 2F below shows the comparison between the highest-scoring word group resulting from the manual review of the entire consultation and the highest-scoring word group resulting from the manual review of the top ten highest-probability word groups identified by the selected random forest model. Each cell indicates the number of consultations where the highest-scoring word group was the specific score indicated by the column and row headers of that cell. The ability of the selected random forest model to identify the overall quality of risk communication for urinary incontinence as measured by manual review across the entire consultation showed a 69.2% accuracy, a 72.9% sensitivity, and a 93.3% specificity.

TABLE 2F

Truth

| | | 0 | 1 | 2 | 3 | 4 | 5 |
|---|---|---|---|---|---|---|---|
| Prediction | 0 | 2 | 0 | 0 | 0 | 0 | 0 |
| | 1 | 0 | 0 | 0 | 0 | 1 | 0 |
| | 2 | 0 | 0 | 4 | 1 | 1 | 0 |
| | 3 | 0 | 0 | 0 | 2 | 1 | 0 |
| | 4 | 0 | 0 | 0 | 0 | 1 | 0 |
| | 5 | 0 | 0 | 0 | 0 | 0 | 0 |

### Example 3

In the third example, a plurality of random forest models were generated to identify word groups (e.g., word groups) related to erectile dysfunction. There were a total of 17,195 word groups among the 42 transcripts, with 336 word groups related to erectile dysfunction and 16,859 word groups not related to erectile dysfunction. 75% of the data (12,896 word groups with 251 related to erectile dysfunction) was used as the training dataset, and 25% of the data (4,299 word groups with 85 related to erectile dysfunction) was used as the internal validation dataset. The plurality of random forest models (each including a certain number of decision trees and a certain number of tokens searched for by each decision trees) is shown in Table 3A below, along with the mean and standard error for both the accuracy of the random forest models (percentage of all determinations that are true) and the area under the ROC curve of the random forest models.

TABLE 3A

| Decision Trees | Tokens | Metric | Mean | Standard Error |
|---|---|---|---|---|
| 100 | 500 | accuracy | 0.92509 | 0.00647 |
| 100 | 500 | roc_auc | 0.94644 | 0.01011 |
| 2575 | 500 | accuracy | 0.93192 | 0.00623 |
| 2575 | 500 | roc_auc | 0.94836 | 0.00982 |
| 5050 | 500 | accuracy | 0.93238 | 0.00613 |
| 5050 | 500 | roc_auc | 0.94771 | 0.01004 |
| 7525 | 500 | accuracy | 0.93207 | 0.00628 |
| 7525 | 500 | roc_auc | 0.94834 | 0.00998 |
| 10000 | 500 | accuracy | 0.93223 | 0.00638 |
| 10000 | 500 | roc_auc | 0.94831 | 0.01007 |
| 100 | 2875 | accuracy | 0.94541 | 0.00536 |
| 100 | 2875 | roc_auc | 0.95684 | 0.00871 |
| 2575 | 2875 | accuracy | 0.9475 | 0.0049 |
| 2575 | 2875 | roc_auc | 0.95598 | 0.00917 |
| 5050 | 2875 | accuracy | 0.9475 | 0.00502 |
| 5050 | 2875 | roc_auc | 0.9564 | 0.00897 |
| 7525 | 2875 | accuracy | 0.94843 | 0.00485 |
| 7525 | 2875 | roc_auc | 0.95596 | 0.00912 |
| 10000 | 2875 | accuracy | 0.94828 | 0.00471 |
| 10000 | 2875 | roc_auc | 0.95651 | 0.00895 |
| 100 | 5250 | accuracy | 0.94456 | 0.00386 |
| 100 | 5250 | roc_auc | 0.95397 | 0.00933 |
| 2575 | 5250 | accuracy | 0.94867 | 0.00521 |
| 2575 | 5250 | roc_auc | 0.95646 | 0.00904 |
| 5050 | 5250 | accuracy | 0.94867 | 0.00456 |
| 5050 | 5250 | roc_auc | 0.95603 | 0.00898 |
| 7525 | 5250 | accuracy | 0.94789 | 0.00458 |
| 7525 | 5250 | roc_auc | 0.95617 | 0.00897 |
| 10000 | 5250 | accuracy | 0.94797 | 0.00463 |
| 10000 | 5250 | roc_auc | 0.95616 | 0.00902 |
| 100 | 7625 | accuracy | 0.94425 | 0.00464 |
| 100 | 7625 | roc_auc | 0.95425 | 0.00919 |
| 2575 | 7625 | accuracy | 0.94851 | 0.00479 |
| 2575 | 7625 | roc_auc | 0.95598 | 0.00897 |
| 5050 | 7625 | accuracy | 0.94789 | 0.00477 |
| 5050 | 7625 | roc_auc | 0.95585 | 0.00915 |
| 7525 | 7625 | accuracy | 0.94766 | 0.00477 |
| 7525 | 7625 | roc_auc | 0.95647 | 0.0089 |
| 10000 | 7625 | accuracy | 0.94797 | 0.00443 |
| 10000 | 7625 | roc_auc | 0.95609 | 0.00896 |
| 100 | 10000 | accuracy | 0.94246 | 0.0059 |
| 100 | 10000 | roc_auc | 0.95609 | 0.00813 |
| 2575 | 10000 | accuracy | 0.94688 | 0.00524 |
| 2575 | 10000 | roc_auc | 0.95562 | 0.00899 |
| 5050 | 10000 | accuracy | 0.94859 | 0.00484 |
| 5050 | 10000 | roc_auc | 0.95581 | 0.00912 |
| 7525 | 10000 | accuracy | 0.94828 | 0.00482 |
| 7525 | 10000 | roc_auc | 0.95631 | 0.00896 |
| 10000 | 10000 | accuracy | 0.94789 | 0.00505 |
| 10000 | 10000 | roc_auc | 0.9562 | 0.00898 |

The random forest model selected for the predetermined topic of erectile dysfunction is the random forest model with the highest mean area under the ROC curve, which includes 100 decision trees and 2,875 tokens. Table 3B below shows the top 100 tokens for this random forest model when validated using the internal validation dataset.

TABLE 3B

| Rank | Variable | Importance |
|---|---|---|
| 1 | tfidf_sentences_erectil | 17.82 |
| 2 | tfidf_sentences_erect | 12.98 |
| 3 | tfidf_sentences_function | 10.47 |
| 4 | tfidf_sentences_nerv | 8.09 |
| 5 | tfidf_sentences_dysfunct | 6.97 |
| 6 | tfidf_sentences_can | 3.47 |
| 7 | tfidf_sentences_contin | 3.39 |
| 8 | tfidf_sentences_potenc | 3.31 |
| 9 | tfidf_sentences_men | 3.2 |
| 10 | tfidf_sentences_urinari | 3.16 |
| 11 | tfidf_sentences_surgeri | 2.47 |
| 12 | tfidf_sentences_sexual | 2.3 |
| 13 | tfidf_sentences_percent | 2.29 |
| 14 | tfidf_sentences_issu | 2.26 |
| 15 | tfidf_sentences_it' | 2.09 |
| 16 | tfidf_sentences_thei | 2.06 |
| 17 | tfidf_sentences_peni | 1.99 |
| 18 | tfidf_sentences_impot | 1.92 |
| 19 | tfidf_sentences_viagra | 1.75 |
| 20 | tfidf_sentences_baselin | 1.68 |
| 21 | tfidf_sentences_okai | 1.67 |
| 22 | tfidf_sentences_year | 1.62 |
| 23 | tfidf_sentences_intact | 1.6 |
| 24 | tfidf_sentences_month | 1.59 |
| 25 | tfidf_sentences_ar | 1.53 |
| 26 | tfidf_sentences_ag | 1.52 |
| 27 | tfidf_sentences_work | 1.48 |
| 28 | tfidf_sentences_first | 1.46 |
| 29 | tfidf_sentences_come | 1.39 |
| 30 | tfidf_sentences_radiat | 1.34 |
| 31 | tfidf_sentences_irrit | 1.31 |
| 32 | tfidf_sentences_get | 1.22 |
| 33 | tfidf_sentences_inject | 1.2 |
| 34 | tfidf_sentences_never | 1.19 |
| 35 | tfidf_sentences_incontin | 1.19 |
| 36 | tfidf_sentences_recov | 1.12 |
| 37 | tfidf_sentences_back | 1.1 |
| 38 | tfidf_sentences_cancer | 1.03 |
| 39 | tfidf_sentences_term | 0.98 |
| 40 | tfidf_sentences_better | 0.97 |
| 41 | tfidf_sentences_import | 0.97 |
| 42 | tfidf_sentences_patient | 0.94 |
| 43 | tfidf_sentences_thing | 0.93 |
| 44 | tfidf_sentences_control | 0.93 |
| 45 | tfidf_sentences_think | 0.88 |
| 46 | tfidf_sentences_health | 0.87 |
| 47 | tfidf_sentences_like | 0.87 |
| 48 | tfidf_sentences_want | 0.84 |
| 49 | tfidf_sentences_also | 0.84 |
| 50 | tfidf_sentences_time | 0.83 |
| 51 | tfidf_sentences_go | 0.82 |
| 52 | tfidf_sentences_right | 0.81 |
| 53 | tfidf_sentences_becaus | 0.8 |
| 54 | tfidf_sentences_still | 0.8 |
| 55 | tfidf_sentences_affect | 0.8 |
| 56 | tfidf_sentences_make | 0.77 |
| 57 | tfidf_sentences_spare | 0.72 |
| 58 | tfidf_sentences_likelihood | 0.7 |
| 59 | tfidf_sentences_correct | 0.68 |
| 60 | tfidf_sentences_damag | 0.68 |
| 61 | tfidf_sentences_take | 0.67 |
| 62 | tfidf_sentences_sensat | 0.65 |
| 63 | tfidf_sentences_prostat | 0.62 |
| 64 | tfidf_sentences_it'll | 0.61 |
| 65 | tfidf_sentences_you'r | 0.59 |
| 66 | tfidf_sentences_beam | 0.59 |
| 67 | tfidf_sentences_predict | 0.57 |
| 68 | tfidf_sentences_run | 0.56 |
| 69 | tfidf_sentences_good | 0.56 |
| 70 | tfidf_sentences_now | 0.55 |
| 71 | tfidf_sentences_question | 0.55 |
| 72 | tfidf_sentences_fact | 0.54 |
| 73 | tfidf_sentences_leakag | 0.54 |
| 74 | tfidf_sentences_recoveri | 0.53 |
| 75 | tfidf_sentences_biopsi | 0.52 |
| 76 | tfidf_sentences_normal | 0.52 |
| 77 | tfidf_sentences_obvious | 0.51 |
| 78 | tfidf_sentences_know | 0.51 |
| 79 | tfidf_sentences_return | 0.5 |
| 80 | tfidf_sentences_probabl | 0.5 |
| 81 | tfidf_sentences_fine | 0.49 |
| 82 | tfidf_sentences_rate | 0.48 |
| 83 | tfidf_sentences_therapi | 0.48 |
| 84 | tfidf_sentences_treat | 0.48 |
| 85 | tfidf_sentences_yeah | 0.48 |
| 86 | tfidf_sentences_urin | 0.48 |
| 87 | tfidf_sentences_risk | 0.47 |
| 88 | tfidf_sentences_todai | 0.47 |
| 89 | tfidf_sentences_gui | 0.47 |
| 90 | tfidf_sentences_around | 0.46 |
| 91 | tfidf_sentences_prosthesi | 0.45 |
| 92 | tfidf_sentences_firm | 0.45 |
| 93 | tfidf_sentences_well | 0.45 |
| 94 | tfidf_sentences_problem | 0.44 |
| 95 | tfidf_sentences_less | 0.44 |
| 96 | tfidf_sentences_help | 0.43 |
| 97 | tfidf_sentences_qualiti | 0.43 |
| 98 | tfidf_sentences_potent | 0.42 |
| 99 | tfidf_sentences_that' | 0.42 |
| 100 | tfidf_sentences_alon | 0.42 |

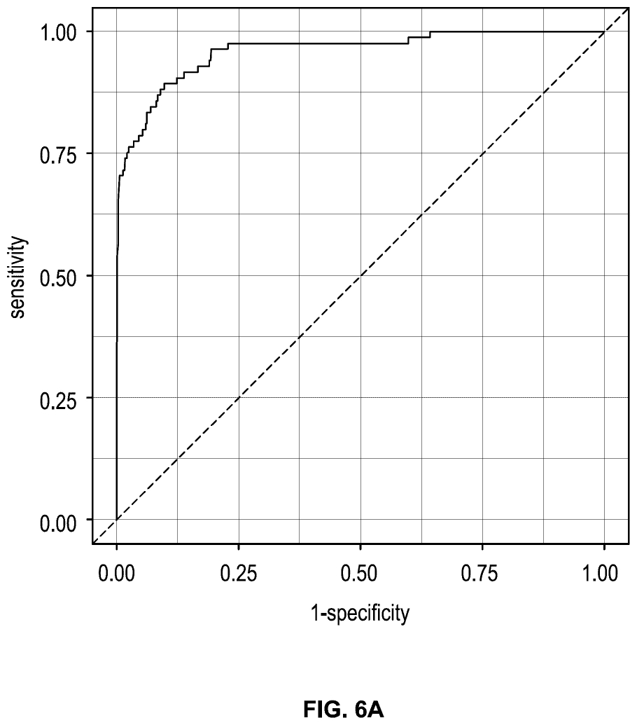

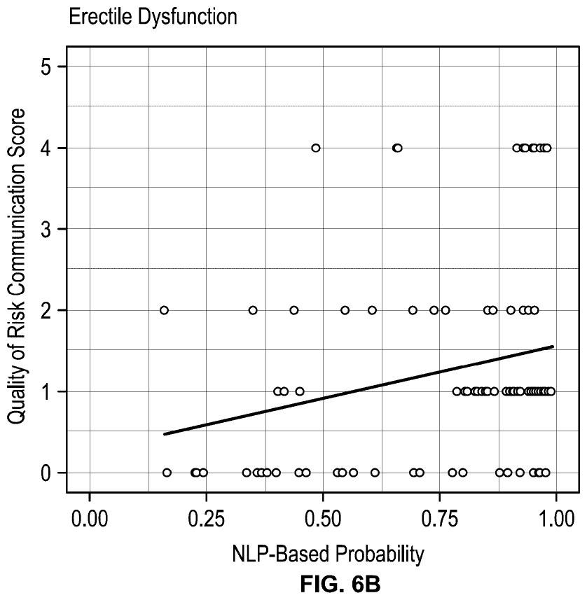

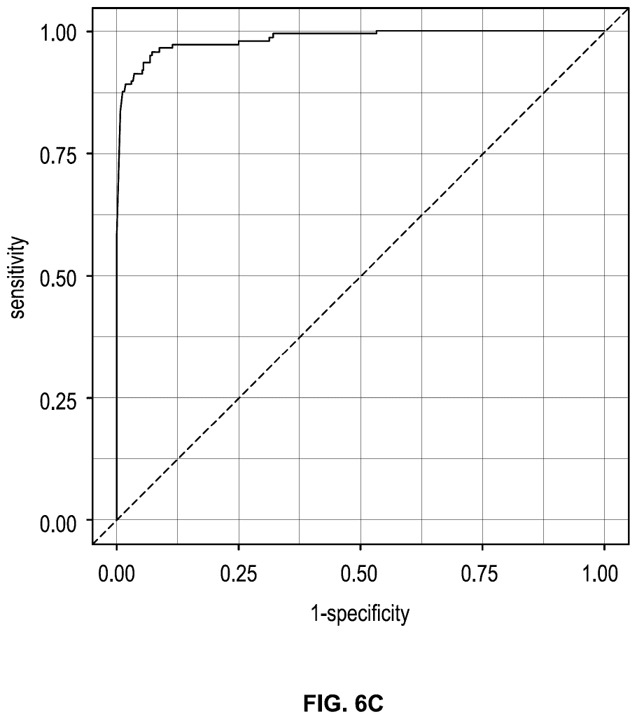

FIG. 6A illustrates the ROC curve for the determination of word groups related to erectile dysfunction, plotted against a reference line indicating an equal rate of true positives and false positives. The area under this ROC curve is 0.9595. Table 3C below shows the value of various different statistical parameters of the selected random forest model for different probability thresholds. As the probability threshold increases (e.g., as the threshold for what is expected to be a determination that a given word group is associated with the predetermined topic becomes more restrictive), the sensitivity decreases while the specificity increases. Conversely, as the threshold probability decreases (e.g., as the threshold for what is expected to be a determination that a given word group is associated with the predetermined topic more inclusive), the sensitivity increases while the specificity decreases. The threshold probability that maximizes the sensitivity and specificity by the J-index is 0.5.

TABLE 3C

| Threshold | TN | FP | FN | TP | accuracy | sens | spec | ppv | npv | j_index |
|---|---|---|---|---|---|---|---|---|---|---|
| 0.5 | 3992 | 222 | 18 | 67 | 0.944 | 0.788 | 0.947 | 0.232 | 0.996 | 0.736 |
| 0.6 | 4126 | 88 | 22 | 63 | 0.974 | 0.741 | 0.979 | 0.417 | 0.995 | 0.72 |
| 0.7 | 4183 | 31 | 25 | 60 | 0.987 | 0.706 | 0.993 | 0.659 | 0.994 | 0.699 |
| 0.8 | 4204 | 10 | 40 | 45 | 0.988 | 0.529 | 0.998 | 0.818 | 0.991 | 0.527 |
| 0.9 | 4212 | 2 | 61 | 24 | 0.985 | 0.282 | 1 | 0.923 | 0.986 | 0.282 |

However, the selection of the ideal probability threshold for the random forest model would also be informed by the purpose of the information. If the intent is to provide a list of the highest yield information but least inclusive information on erectile dysfunction (e.g., containing few word groups that are irrelevant to erectile dysfunction at the cost of missing all the word groups having to do with erectile dysfunction), then a high probability threshold would be selected. If the intent is to provide a list of the most inclusive but less precise information on erectile dysfunction (e.g., containing some word groups that may have less to do with erectile dysfunction but capturing more word groups relevant to erectile dysfunction), then a low probability threshold would be selected.

For each of the 42 consultations, the top ten word groups by probability of being related to erectile dysfunction were used to predict the overall quality of risk communication across the entire consultation using an a priori defined hierarchy for quality assessment. This hierarchy designates quality across a spectrum of increasing granularity of communication: (0) not mentioned, (1) name only (without risk quantification), (2) generalization (“high”/“low”), (3) average percent incidence without timepoint, (4) average percent incidence with timepoint, (5) precision estimate accounting for patient-level characteristics. For each consultation, a reviewer manually analyzed the entire consultation and gave the consultation a score within the hierarchy, where the score generally indicated the highest-scoring word group of all word groups within that consultation. Similarly, a reviewer manually analyzed the top ten word groups identified by selected random forest model and gave the consultation a score within the hierarchy, where the score indicated the highest-scoring word group of the top ten highest-probability word groups. Table 3D below shows the comparison between the highest-scoring word group resulting from the manual review of the entire consultation and the highest-scoring word group resulting from the manual review of the top ten highest-probability word groups identified by the selected random forest model. Each cell indicates the number of consultations where the highest-scoring word group was the specific score indicated by the column and row headers of that cell. The ability of the selected random forest model to identify the overall quality of risk communication for erectile dysfunction as measured by manual review across the entire consultation showed an 88% accuracy, a 91% sensitivity, a 97% specificity, an 83% positive predictive value, and a 97% negative predictive value.

TABLE 3D

Truth

| | | 0 | 1 | 2 | 3 | 4 | 5 |
|---|---|---|---|---|---|---|---|
| Prediction | 0 | 8 | 0 | 0 | 0 | 0 | 0 |
| | 1 | 1 | 8 | 0 | 0 | 0 | 0 |
| | 2 | 1 | 1 | 3 | 0 | 0 | 0 |
| | 3 | 0 | 0 | 0 | 0 | 0 | 0 |
| | 4 | 0 | 0 | 0 | 0 | 14 | 0 |
| | 5 | 0 | 0 | 0 | 0 | 2 | 4 |

FIG. 6B shows a linear regression plot of the scores given to various word groups for the “erectile dysfunction” topic based on manual review, versus the probability that each respective word group is associated with erectile dysfunction, with an overlaid trend line. As shown by the plot, the probability of being associated with erectile dysfunction is well-correlated with the score given to the word group based on the manual review, indicating that the probability assigned by the model can be used to reliably score a consultation (e.g., grade the quality level of the consultation) with respect to erectile dysfunction.

Once the random forest model was generated to identify word groups related to erectile dysfunction, it was externally validated using a sample of 13 transcripts containing a total of 4,658 sentences. 135 of the sentences were related to erectile dysfunction, and 4,523 sentences were not related to erectile dysfunction. FIG. 6C illustrates the ROC curve for this validation, with an area under the curve of 0.982. The model's sensitivity and specificity were calculated for the prediction of word groups related to urinary incontinence at the probability threshold having the highest J-index (0.5) that was identified during training. The model's performance with the external validation dataset using this probability threshold is shown in Table 4E.

TABLE 3E

| | | | | | accu- | | | Balanced |
|---|---|---|---|---|---|---|---|---|
| Threshold | TN | FP | FN | TP | racy | sens | spec | Accuracy |
| 0.5 | 4228 | 295 | 9 | 126 | 0.935 | 0.933 | 0.935 | 0.934 |

For each consultation in the external validation dataset, the top ten word groups (e.g., sentences or phrases spoken by the healthcare provider) by probability of being related to erectile dysfunction were used to predict the overall quality of risk communication across the entire consultation, using the same hierarchy described above. Table 3F below shows the comparison between the highest-scoring word group resulting from the manual review of the entire consultation and the highest-scoring word group resulting from the manual review of the top ten highest-probability word groups identified by the selected random forest model. Each cell indicates the number of consultations where the highest-scoring word group was the specific score indicated by the column and row headers of that cell. The ability of the selected random forest model to identify the overall quality of risk communication for urinary incontinence as measured by manual review across the entire consultation showed a 92.3% accuracy, a 91.7% sensitivity, and a 98.7% specificity.

TABLE 3F

Truth

| | | 0 | 1 | 2 | 3 | 4 | 5 |
|---|---|---|---|---|---|---|---|
| Prediction | 0 | 0 | 0 | 0 | 0 | 0 | 0 |
| | 1 | 0 | 0 | 1 | 0 | 0 | 0 |
| | 2 | 0 | 0 | 2 | 0 | 0 | 0 |
| | 3 | 0 | 0 | 0 | 1 | 0 | 0 |
| | 4 | 0 | 0 | 0 | 0 | 3 | 0 |
| | 5 | 0 | 0 | 0 | 0 | 0 | 6 |

### Example 4

In the fourth example, a plurality of random forest models were generated to identify word groups (e.g., word groups) related to life expectancy. There were a total of 17,195 word groups among the 42 transcripts, with 88 word groups related to life expectancy and 17,107 word groups not related to life expectancy. 75% of the data (12,896 word groups with 67 related to life expectancy) was used as the training dataset, and 25% of the data (4,299 word groups with 21 related to life expectancy) was used as the internal validation dataset. The plurality of random forest models (each including a certain number of decision trees and a certain number of tokens searched for by each decision trees) is shown in Table 4A below, along with the mean and standard error for both the accuracy of the random forest models (percentage of all determinations that are true) and the area under the ROC curve of the random forest models.

TABLE 4A

| Decision Trees | Tokens | Metric | Mean | Standard Error |
|---|---|---|---|---|
| 100 | 500 | accuracy | 0.91873 | 0.00474 |
| 100 | 500 | roc_auc | 0.89706 | 0.01984 |
| 2575 | 500 | accuracy | 0.92292 | 0.00425 |
| 2575 | 500 | roc_auc | 0.89923 | 0.01826 |
| 5050 | 500 | accuracy | 0.92315 | 0.00448 |
| 5050 | 500 | roc_auc | 0.8993 | 0.018 |
| 7525 | 500 | accuracy | 0.92377 | 0.00476 |
| 7525 | 500 | roc_auc | 0.89935 | 0.01803 |
| 10000 | 500 | accuracy | 0.92346 | 0.0046 |
| 10000 | 500 | roc_auc | 0.899 | 0.0186 |
| 100 | 2875 | accuracy | 0.9171 | 0.00465 |
| 100 | 2875 | roc_auc | 0.89514 | 0.01877 |
| 2575 | 2875 | accuracy | 0.92579 | 0.00341 |
| 2575 | 2875 | roc_auc | 0.89927 | 0.01874 |
| 5050 | 2875 | accuracy | 0.92656 | 0.00347 |
| 5050 | 2875 | roc_auc | 0.90103 | 0.0187 |
| 7525 | 2875 | accuracy | 0.92602 | 0.00367 |
| 7525 | 2875 | roc_auc | 0.901 | 0.01911 |
| 10000 | 2875 | accuracy | 0.92594 | 0.0034 |
| 10000 | 2875 | roc_auc | 0.90052 | 0.01888 |
| 100 | 5250 | accuracy | 0.9209 | 0.00382 |
| 100 | 5250 | roc_auc | 0.87721 | 0.02235 |
| 2575 | 5250 | accuracy | 0.92385 | 0.00339 |
| 2575 | 5250 | roc_auc | 0.90185 | 0.01851 |
| 5050 | 5250 | accuracy | 0.92362 | 0.00322 |
| 5050 | 5250 | roc_auc | 0.9021 | 0.01897 |
| 7525 | 5250 | accuracy | 0.92532 | 0.00331 |
| 7525 | 5250 | roc_auc | 0.90233 | 0.0183 |
| 10000 | 5250 | accuracy | 0.92393 | 0.00366 |
| 10000 | 5250 | roc_auc | 0.90168 | 0.019 |
| 100 | 7625 | accuracy | 0.91804 | 0.00434 |
| 100 | 7625 | roc_auc | 0.89518 | 0.02156 |
| 2575 | 7625 | accuracy | 0.92308 | 0.00323 |
| 2575 | 7625 | roc_auc | 0.90004 | 0.01969 |
| 5050 | 7625 | accuracy | 0.92478 | 0.00342 |
| 5050 | 7625 | roc_auc | 0.90274 | 0.01858 |
| 7525 | 7625 | accuracy | 0.92385 | 0.00346 |
| 7525 | 7625 | roc_auc | 0.90186 | 0.01882 |
| 10000 | 7625 | accuracy | 0.92432 | 0.00351 |
| 10000 | 7625 | roc_auc | 0.90126 | 0.01885 |
| 100 | 10000 | accuracy | 0.9133 | 0.00546 |
| 100 | 10000 | roc_auc | 0.89471 | 0.01867 |
| 2575 | 10000 | accuracy | 0.92331 | 0.00346 |
| 2575 | 10000 | roc_auc | 0.90164 | 0.01873 |
| 5050 | 10000 | accuracy | 0.92501 | 0.00359 |
| 5050 | 10000 | roc_auc | 0.90111 | 0.01929 |
| 7525 | 10000 | accuracy | 0.92432 | 0.00332 |
| 7525 | 10000 | roc_auc | 0.90251 | 0.01878 |
| 10000 | 10000 | accuracy | 0.92439 | 0.00358 |
| 10000 | 10000 | roc_auc | 0.90126 | 0.01879 |

The random forest model selected for the predetermined topic of life expectancy is the random forest model with the highest mean area under the ROC curve, which includes 5,000 decision trees and 7,625 tokens. Table 4B below shows the top 100 tokens for this random forest model when validated using the internal validation dataset.

TABLE 4B

| Rank | Variable | Importance |
|---|---|---|
| 1 | tfidf_sentences_year | 3.97 |
| 2 | tfidf_sentences_live | 1.8 |
| 3 | tfidf_sentences_life | 1.13 |
| 4 | tfidf_sentences_expect | 0.84 |
| 5 | tfidf_sentences_you'r | 0.8 |
| 6 | tfidf_sentences_plan | 0.73 |
| 7 | tfidf_sentences_cancer | 0.54 |
| 8 | tfidf_sentences_longer | 0.52 |
| 9 | tfidf_sentences_ahead | 0.51 |
| 10 | tfidf_sentences_long | 0.48 |
| 11 | tfidf_sentences_ag | 0.46 |
| 12 | tfidf_sentences_got | 0.45 |
| 13 | tfidf_sentences_think | 0.44 |
| 14 | tfidf_sentences_go | 0.41 |
| 15 | tfidf_sentences_need | 0.41 |
| 16 | tfidf_sentences_even | 0.39 |
| 17 | tfidf_sentences_take | 0.39 |
| 18 | tfidf_sentences_know | 0.38 |
| 19 | tfidf_sentences_okai | 0.37 |
| 20 | tfidf_sentences_guess | 0.37 |
| 21 | tfidf_sentences_look | 0.32 |
| 22 | tfidf_sentences_that' | 0.31 |
| 23 | tfidf_sentences_hope | 0.3 |
| 24 | tfidf_sentences_next | 0.29 |
| 25 | tfidf_sentences_healthi | 0.29 |
| 26 | tfidf_sentences_man | 0.29 |
| 27 | tfidf_sentences_now | 0.29 |
| 28 | tfidf_sentences_chanc | 0.28 |
| 29 | tfidf_sentences_yeah | 0.27 |
| 30 | tfidf_sentences_you'v | 0.27 |
| 31 | tfidf_sentences_someth | 0.27 |
| 32 | tfidf_sentences_patient | 0.26 |
| 33 | tfidf_sentences_care | 0.24 |
| 34 | tfidf_sentences_it' | 0.23 |
| 35 | tfidf_sentences_old | 0.22 |
| 36 | tfidf_sentences_around | 0.22 |
| 37 | tfidf_sentences_thi | 0.22 |
| 38 | tfidf_sentences_health | 0.22 |
| 39 | tfidf_sentences_like | 0.21 |
| 40 | tfidf_sentences_we'r | 0.21 |
| 41 | tfidf_sentences_sai | 0.2 |
| 42 | tfidf_sentences_ar | 0.2 |
| 43 | tfidf_sentences_twenti | 0.19 |
| 44 | tfidf_sentences_tumor | 0.19 |
| 45 | tfidf_sentences_averag | 0.19 |
| 46 | tfidf_sentences_prostat | 0.19 |
| 47 | tfidf_sentences_urin | 0.19 |
| 48 | tfidf_sentences_right | 0.18 |
| 49 | tfidf_sentences_good | 0.17 |
| 50 | tfidf_sentences_less | 0.17 |
| 51 | tfidf_sentences_exactli | 0.16 |
| 52 | tfidf_sentences_pure | 0.16 |
| 53 | tfidf_sentences_want | 0.15 |
| 54 | tfidf_sentences_gui | 0.14 |
| 55 | tfidf_sentences_still | 0.14 |
| 56 | tfidf_sentences_eight | 0.13 |
| 57 | tfidf_sentences_watch | 0.13 |
| 58 | tfidf_sentences_high | 0.13 |
| 59 | tfidf_sentences_lot | 0.13 |
| 60 | tfidf_sentences_come | 0.13 |
| 61 | tfidf_sentences_plu | 0.12 |
| 62 | tfidf_sentences_treatment | 0.12 |
| 63 | tfidf_sentences_thing | 0.12 |
| 64 | tfidf_sentences_veri | 0.12 |
| 65 | tfidf_sentences_ha | 0.12 |
| 66 | tfidf_sentences_just | 0.12 |
| 67 | tfidf_sentences_becaus | 0.11 |
| 68 | tfidf_sentences_peopl | 0.11 |
| 69 | tfidf_sentences_surgeri | 0.11 |
| 70 | tfidf_sentences_term | 0.11 |
| 71 | tfidf_sentences_grandchildren | 0.11 |
| 72 | tfidf_sentences_risk | 0.11 |
| 73 | tfidf_sentences_feel | 0.1 |
| 74 | tfidf_sentences_much | 0.1 |
| 75 | tfidf_sentences_enough | 0.1 |
| 76 | tfidf_sentences_aggress | 0.1 |
| 77 | tfidf_sentences_ani | 0.1 |
| 78 | tfidf_sentences_mani | 0.1 |
| 79 | tfidf_sentences_comorbid | 0.1 |
| 80 | tfidf_sentences_they'r | 0.09 |
| 81 | tfidf_sentences_pretti | 0.09 |
| 82 | tfidf_sentences_grow | 0.09 |
| 83 | tfidf_sentences_major | 0.09 |
| 84 | tfidf_sentences_men | 0.09 |
| 85 | tfidf_sentences_littl | 0.08 |
| 86 | tfidf_sentences_american | 0.08 |
| 87 | tfidf_sentences_suggest | 0.08 |
| 88 | tfidf_sentences_month | 0.08 |
| 89 | tfidf_sentences_di | 0.08 |
| 90 | tfidf_sentences_therapi | 0.08 |
| 91 | tfidf_sentences_make | 0.08 |
| 92 | tfidf_sentences_statist | 0.08 |
| 93 | tfidf_sentences_perfect | 0.08 |
| 94 | tfidf_sentences_u. | 0.08 |
| 95 | tfidf_sentences_everybodi | 0.08 |
| 96 | tfidf_sentences_thought | 0.08 |
| 97 | tfidf_sentences_intermedi | 0.08 |
| 98 | tfidf_sentences_alon | 0.07 |
| 99 | tfidf_sentences_likelihood | 0.07 |
| 100 | tfidf_sentences_base | 0.07 |

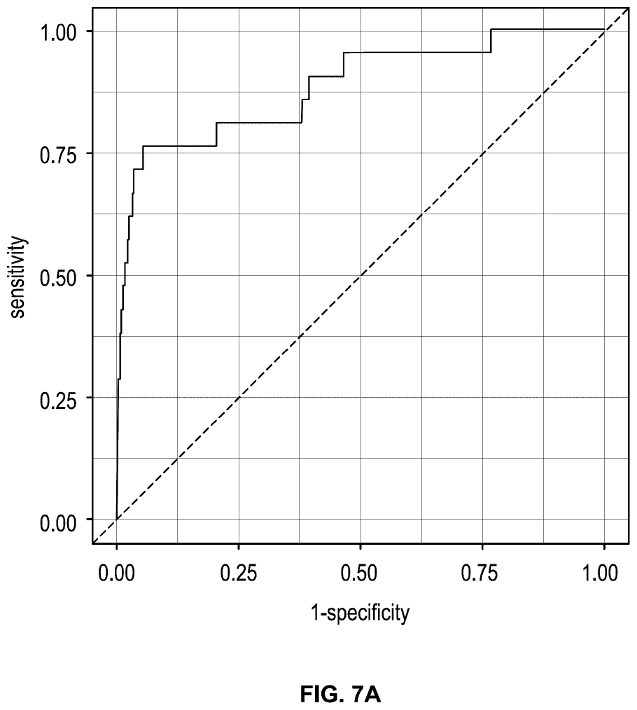

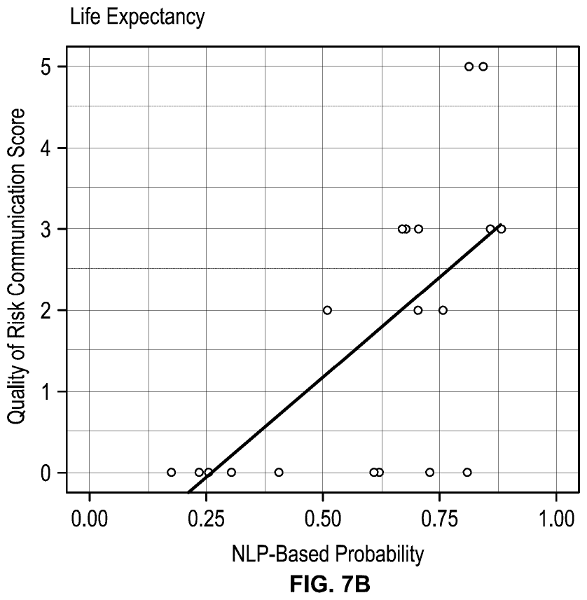

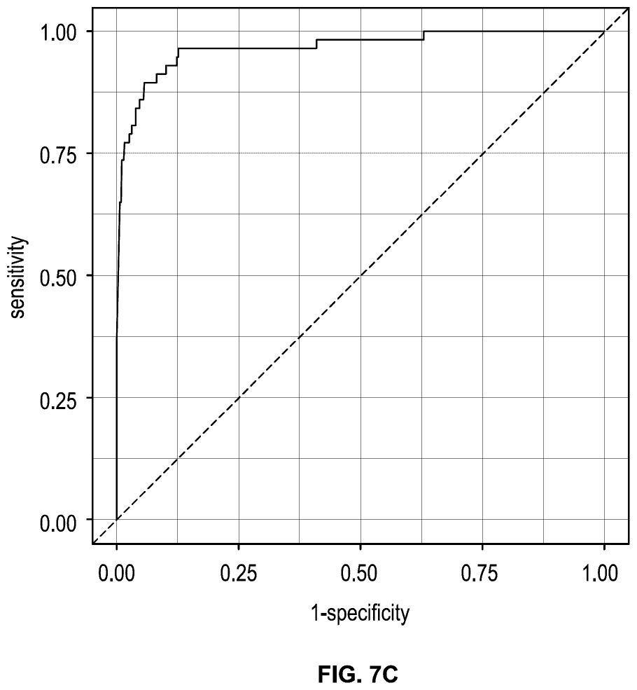

FIG. 7A illustrates the ROC curve for the determination of word groups related to life expectancy, plotted against a reference line indicating an equal rate of true positives and false positives. The area under this ROC curve is 0.8827. Table 4C below shows the value of various different statistical parameters of the selected random forest model for different probability thresholds. As the probability threshold increases (e.g., as the threshold for what is expected to be a determination that a given word group is associated with the predetermined topic becomes more restrictive), the sensitivity decreases while the specificity increases. Conversely, as the threshold probability decreases (e.g., as the threshold for what is expected to be a determination that a given word group is associated with the predetermined topic more inclusive), the sensitivity increases while the specificity decreases. The threshold probability that maximizes the sensitivity and specificity by the J-index is 0.5.

TABLE 4C

| Threshold | TN | FP | FN | TP | accuracy | sens | spec | ppv | npv | j_index |
|---|---|---|---|---|---|---|---|---|---|---|
| 0.5 | 3969 | 309 | 5 | 16 | 0.927 | 0.762 | 0.928 | 0.049 | 0.999 | 0.69 |
| 0.6 | 4209 | 69 | 11 | 10 | 0.981 | 0.476 | 0.984 | 0.127 | 0.997 | 0.46 |
| 0.7 | 4262 | 16 | 18 | 3 | 0.992 | 0.143 | 0.996 | 0.158 | 0.996 | 0.139 |
| 0.8 | 4277 | 1 | 21 | 0 | 0.995 | 0 | 1 | 0 | 0.995 | 0 |
| 0.9 | 4278 | 0 | 21 | 0 | 0.995 | 0 | 1 | NA | 0.995 | 0 |

However, the selection of the ideal probability threshold for the random forest model would also be informed by the purpose of the information. If the intent is to provide a list of the highest yield information but least inclusive information on life expectancy (e.g., containing few word groups that are irrelevant to life expectancy at the cost of missing all the word groups having to do with life expectancy), then a high probability threshold would be selected. If the intent is to provide a list of the most inclusive but less precise information on life expectancy (e.g., containing some word groups that may have less to do with life expectancy but capturing more word groups relevant to life expectancy), then a low probability threshold would be selected.

For each of the 42 consultations, the top ten word groups by probability of being related to life expectancy were used to predict the overall quality of risk communication across the entire consultation using an a priori defined hierarchy for quality assessment. This hierarchy designates quality across a spectrum of increasing granularity of communication: (0) not mentioned, (1) name only (without risk quantification), (2) generalization (“high”/“low”), (3) average percent incidence without timepoint, (4) average percent incidence with timepoint, (5) precision estimate accounting for patient-level characteristics. For each consultation, a reviewer manually analyzed the entire consultation and gave the consultation a score within the hierarchy, where the score generally indicated the highest-scoring word group of all word groups within that consultation. Similarly, a reviewer manually analyzed the top ten word groups identified by selected random forest model and gave the consultation a score within the hierarchy, where the score indicated the highest-scoring word group of the top ten highest-probability word groups. Table 4D below shows the comparison between the highest-scoring word group resulting from the manual review of the entire consultation and the highest-scoring word group resulting from the manual review of the top ten highest-probability word groups identified by the selected random forest model. Each cell indicates the number of consultations where the highest-scoring word group was the specific score indicated by the column and row headers of that cell. The ability of the selected random forest model to identify the overall quality of risk communication for life expectancy as measured by manual review across the entire consultation showed a 62% accuracy, a 54% sensitivity, a 90% specificity, and a negative predictive value of 91%.

TABLE 4D

Truth

| | | 0 | 1 | 2 | 3 | 4 | 5 |
|---|---|---|---|---|---|---|---|
| Prediction | 0 | 5 | 4 | 3 | 2 | 1 | 5 |
| | 1 | 0 | 3 | 1 | 0 | 0 | 0 |
| | 2 | 1 | 0 | 9 | 1 | 2 | 1 |
| | 3 | 0 | 0 | 0 | 0 | 0 | 0 |
| | 4 | 0 | 0 | 0 | 1 | 9 | 0 |
| | 5 | 5 | 4 | 3 | 2 | 1 | 5 |

FIG. 7B shows a linear regression plot of the scores given to various word groups for the “life expectancy” topic based on manual review, versus the probability that each respective word group is associated with life expectancy, with an overlaid trend line. As shown by the plot, the probability of being associated with life expectancy is well-correlated with the score given to the word group based on the manual review, indicating that the probability assigned by the model can be used to reliably score a consultation (e.g., grade the quality level of the consultation) with respect to life expectancy.

Once the random forest model was generated to identify word groups related to life expectancy, it was externally validated using a sample of 13 transcripts containing a total of 4,658 sentences. 57 of the sentences were related to life expectancy, and 4,601 sentences were not related to life expectancy. FIG. 7C illustrates the ROC curve for this validation, with an area under the curve of 0.967. The model's sensitivity and specificity were calculated for the prediction of word groups related to life expectancy at the probability threshold having the highest J-index (0.5) that was identified during training. The model's performance with the external validation dataset using this probability threshold is shown in Table 1E.

TABLE 4E

| | | | | | | | | Balanced |
|---|---|---|---|---|---|---|---|---|
| Threshold | TN | FP | FN | TP | accuracy | sens | spec | Accuracy |
| 0.5 | 4311 | 290 | 6 | 51 | 0.936 | 0.895 | 0.937 | 0.916 |

For each consultation in the external validation dataset, the top ten word groups (e.g., sentences or phrases spoken by the healthcare provider) by probability of being related to life expectancy were used to predict the overall quality of risk communication across the entire consultation, using the same hierarchy described above. Table 4F below shows the comparison between the highest-scoring word group resulting from the manual review of the entire consultation and the highest-scoring word group resulting from the manual review of the top ten highest-probability word groups identified by the selected random forest model. Each cell indicates the number of consultations where the highest-scoring word group was the specific score indicated by the column and row headers of that cell. The ability of the selected random forest model to identify the overall quality of risk communication for urinary incontinence as measured by manual review across the entire consultation showed a 92.3% accuracy, a 96.7% sensitivity, and a 98.7% specificity.

TABLE 4F

Truth

| | | 0 | 1 | 2 | 3 | 4 | 5 |
|---|---|---|---|---|---|---|---|
| Prediction | 0 | 0 | 0 | 0 | 0 | 0 | 0 |
| | 1 | 0 | 0 | 0 | 0 | 0 | 0 |
| | 2 | 0 | 0 | 3 | 0 | 0 | 0 |
| | 3 | 0 | 0 | 0 | 1 | 0 | 0 |
| | 4 | 0 | 0 | 0 | 0 | 9 | 0 |
| | 5 | 0 | 0 | 0 | 0 | 0 | 0 |

### Example 5

In the first example, a plurality of random forest models were generated to identify word groups (e.g., word groups) related to cancer prognosis. There were a total of 17,195 word groups among the 42 transcripts, with 252 word groups related to cancer prognosis and 16,943 word groups not related to cancer prognosis. 75% of the data (12,896 word groups with 178 related to cancer prognosis) was used as the training dataset, and 25% of the data (4,299 word groups with 74 related to cancer prognosis) was used as the internal validation dataset. The plurality of random forest models (each including a certain number of decision trees and a certain number of tokens searched for by each decision trees) is shown in Table 5A below, along with the mean and standard error for both the accuracy of the random forest models (percentage of all determinations that are true) and the area under the ROC curve of the random forest models.

TABLE 5A

| Decision Trees | Tokens | Metric | Mean | Standard Error |
|---|---|---|---|---|
| 100 | 500 | accuracy | 0.844 | 0.0059 |
| 100 | 500 | roc_auc | 0.8752 | 0.0135 |
| 2575 | 500 | accuracy | 0.8494 | 0.0053 |
| 2575 | 500 | roc_auc | 0.8822 | 0.0121 |
| 5050 | 500 | accuracy | 0.8497 | 0.0047 |
| 5050 | 500 | roc_auc | 0.8827 | 0.0124 |
| 7525 | 500 | accuracy | 0.8517 | 0.005 |
| 7525 | 500 | roc_auc | 0.8828 | 0.0122 |
| 10000 | 500 | accuracy | 0.851 | 0.0047 |
| 10000 | 500 | roc_auc | 0.8824 | 0.0123 |
| 100 | 2875 | accuracy | 0.8783 | 0.0054 |
| 100 | 2875 | roc_auc | 0.8895 | 0.0129 |
| 2575 | 2875 | accuracy | 0.8857 | 0.0035 |
| 2575 | 2875 | roc_auc | 0.8918 | 0.0133 |
| 5050 | 2875 | accuracy | 0.888 | 0.0036 |
| 5050 | 2875 | roc_auc | 0.8911 | 0.0135 |
| 7525 | 2875 | accuracy | 0.8873 | 0.0034 |
| 7525 | 2875 | roc_auc | 0.8909 | 0.0135 |
| 10000 | 2875 | accuracy | 0.8868 | 0.0036 |
| 10000 | 2875 | roc_auc | 0.8913 | 0.0134 |
| 100 | 5250 | accuracy | 0.879 | 0.0046 |
| 100 | 5250 | roc_auc | 0.887 | 0.0128 |
| 2575 | 5250 | accuracy | 0.8878 | 0.0033 |
| 2575 | 5250 | roc_auc | 0.8931 | 0.0131 |
| 5050 | 5250 | accuracy | 0.8873 | 0.0034 |
| 5050 | 5250 | roc_auc | 0.8924 | 0.0136 |
| 7525 | 5250 | accuracy | 0.8879 | 0.003 |
| 7525 | 5250 | roc_auc | 0.8926 | 0.0134 |
| 10000 | 5250 | accuracy | 0.8876 | 0.0034 |
| 10000 | 5250 | roc_auc | 0.8927 | 0.0135 |
| 100 | 7625 | accuracy | 0.8862 | 0.0044 |
| 100 | 7625 | roc_auc | 0.8939 | 0.0145 |
| 2575 | 7625 | accuracy | 0.8873 | 0.0029 |
| 2575 | 7625 | roc_auc | 0.8923 | 0.0132 |
| 5050 | 7625 | accuracy | 0.8872 | 0.0032 |
| 5050 | 7625 | roc_auc | 0.8928 | 0.0133 |
| 7525 | 7625 | accuracy | 0.8883 | 0.0031 |
| 7525 | 7625 | roc_auc | 0.8926 | 0.0135 |
| 10000 | 7625 | accuracy | 0.8873 | 0.0034 |
| 10000 | 7625 | roc_auc | 0.8927 | 0.0136 |
| 100 | 10000 | accuracy | 0.8821 | 0.0052 |
| 100 | 10000 | roc_auc | 0.8897 | 0.0123 |
| 2575 | 10000 | accuracy | 0.8869 | 0.0033 |
| 2575 | 10000 | roc_auc | 0.8923 | 0.0133 |
| 5050 | 10000 | accuracy | 0.8876 | 0.0033 |
| 5050 | 10000 | roc_auc | 0.8926 | 0.0133 |
| 7525 | 10000 | accuracy | 0.888 | 0.0032 |
| 7525 | 10000 | roc_auc | 0.8924 | 0.0136 |
| 10000 | 10000 | accuracy | 0.8876 | 0.0035 |
| 10000 | 10000 | roc_auc | 0.8923 | 0.0135 |

The random forest model selected for the predetermined topic of cancer prognosis is the random forest model with the highest mean area under the ROC curve, which includes 100 decision trees and 7,675 tokens. Table 5B below shows the top 100 tokens for this random forest model when validated using the internal validation dataset.

TABLE 5B

| Rank | Variable | Importance |
|---|---|---|
| 1 | tfidf_sentences_percent | 7.49 |
| 2 | tfidf_sentences_cancer | 7.02 |
| 3 | tfidf_sentences_likelihood | 3.27 |
| 4 | tfidf_sentences_thi | 2.93 |
| 5 | tfidf_sentences_risk | 2.92 |
| 6 | tfidf_sentences_year | 2.77 |
| 7 | tfidf_sentences_die | 2.66 |
| 8 | tfidf_sentences_it' | 2.21 |
| 9 | tfidf_sentences_chanc | 1.81 |
| 10 | tfidf_sentences_death | 1.78 |
| 11 | tfidf_sentences_caus | 1.77 |
| 12 | tfidf_sentences_ten | 1.75 |
| 13 | tfidf_sentences_threaten | 1.69 |
| 14 | tfidf_sentences_life | 1.69 |
| 15 | tfidf_sentences_now | 1.68 |
| 16 | tfidf_sentences_dy | 1.4 |
| 17 | tfidf_sentences_noth | 1.35 |
| 18 | tfidf_sentences_still | 1.25 |
| 19 | tfidf_sentences_reduc | 1.17 |
| 20 | tfidf_sentences_cure | 1.09 |
| 21 | tfidf_sentences_harm | 1.06 |
| 22 | tfidf_sentences_even | 1.03 |
| 23 | tfidf_sentences_like | 1.03 |
| 24 | tfidf_sentences_time | 0.95 |
| 25 | tfidf_sentences_yeah | 0.94 |
| 26 | tfidf_sentences_slow | 0.93 |
| 27 | tfidf_sentences_spread | 0.92 |
| 28 | tfidf_sentences_can | 0.88 |
| 29 | tfidf_sentences_we'r | 0.86 |
| 30 | tfidf_sentences_rate | 0.84 |
| 31 | tfidf_sentences_grow | 0.83 |
| 32 | tfidf_sentences_number | 0.82 |
| 33 | tfidf_sentences_prostat | 0.81 |
| 34 | tfidf_sentences_zero | 0.81 |
| 35 | tfidf_sentences_low | 0.78 |
| 36 | tfidf_sentences_treatment | 0.78 |
| 37 | tfidf_sentences_wai | 0.76 |
| 38 | tfidf_sentences_think | 0.73 |
| 39 | tfidf_sentences_live | 0.72 |
| 40 | tfidf_sentences_natur | 0.72 |
| 41 | tfidf_sentences_recur | 0.69 |
| 42 | tfidf_sentences_come | 0.66 |
| 43 | tfidf_sentences_tell | 0.66 |
| 44 | tfidf_sentences_someth | 0.64 |
| 45 | tfidf_sentences_di | 0.63 |
| 46 | tfidf_sentences_said | 0.61 |
| 47 | tfidf_sentences_outsid | 0.61 |
| 48 | tfidf_sentences_happen | 0.59 |
| 49 | tfidf_sentences_wa | 0.59 |
| 50 | tfidf_sentences_ar | 0.58 |
| 51 | tfidf_sentences_radiat | 0.58 |
| 52 | tfidf_sentences_lymph | 0.57 |
| 53 | tfidf_sentences_kill | 0.55 |
| 54 | tfidf_sentences_point | 0.54 |
| 55 | tfidf_sentences_person | 0.53 |
| 56 | tfidf_sentences_you'r | 0.53 |
| 57 | tfidf_sentences_veri | 0.52 |
| 58 | tfidf_sentences_node | 0.51 |
| 59 | tfidf_sentences_back | 0.51 |
| 60 | tfidf_sentences_men | 0.48 |
| 61 | tfidf_sentences_bad | 0.48 |
| 62 | tfidf_sentences_go | 0.46 |
| 63 | tfidf_sentences_surgeri | 0.46 |
| 64 | tfidf_sentences_watch | 0.46 |
| 65 | tfidf_sentences_higher | 0.46 |
| 66 | tfidf_sentences_much | 0.45 |
| 67 | tfidf_sentences_there' | 0.45 |
| 68 | tfidf_sentences_alreadi | 0.45 |
| 69 | tfidf_sentences_almost | 0.43 |
| 70 | tfidf_sentences_term | 0.43 |
| 71 | tfidf_sentences_three | 0.43 |
| 72 | tfidf_sentences_answer | 0.42 |
| 73 | tfidf_sentences_what' | 0.42 |
| 74 | tfidf_sentences_long | 0.42 |
| 75 | tfidf_sentences_get | 0.41 |
| 76 | tfidf_sentences_period | 0.41 |
| 77 | tfidf_sentences_let' | 0.4 |
| 78 | tfidf_sentences_kind | 0.4 |
| 79 | tfidf_sentences_look | 0.4 |
| 80 | tfidf_sentences_thing | 0.4 |
| 81 | tfidf_sentences_fact | 0.39 |
| 82 | tfidf_sentences_onli | 0.39 |
| 83 | tfidf_sentences_tumor | 0.38 |
| 84 | tfidf_sentences_capsul | 0.38 |
| 85 | tfidf_sentences_everybodi | 0.37 |
| 86 | tfidf_sentences_that' | 0.37 |
| 87 | tfidf_sentences_take | 0.37 |
| 88 | tfidf_sentences_hormon | 0.37 |
| 89 | tfidf_sentences_patient | 0.36 |
| 90 | tfidf_sentences_mai | 0.36 |
| 91 | tfidf_sentences_deadli | 0.36 |
| 92 | tfidf_sentences_control | 0.35 |
| 93 | tfidf_sentences_sai | 0.35 |
| 94 | tfidf_sentences_know | 0.35 |
| 95 | tfidf_sentences_keep | 0.35 |
| 96 | tfidf_sentences_becaus | 0.34 |
| 97 | tfidf_sentences_forev | 0.34 |
| 98 | tfidf_sentences_right | 0.34 |
| 99 | tfidf_sentences_nobodi | 0.33 |
| 100 | tfidf_sentences_side | 0.33 |

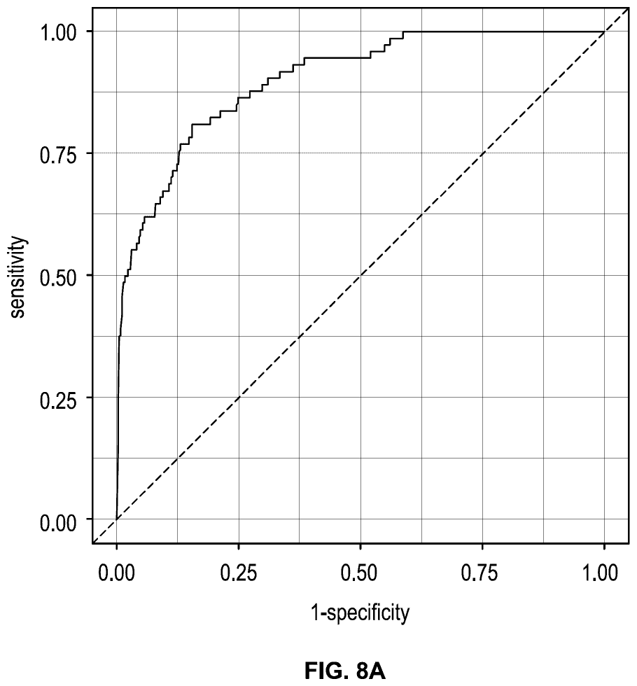

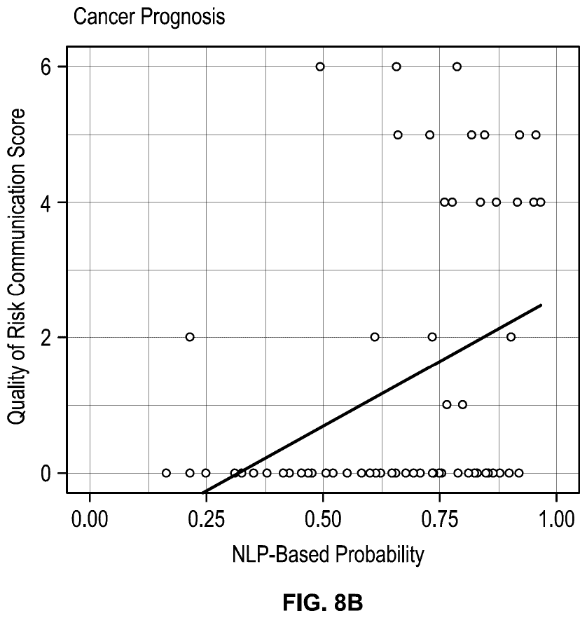

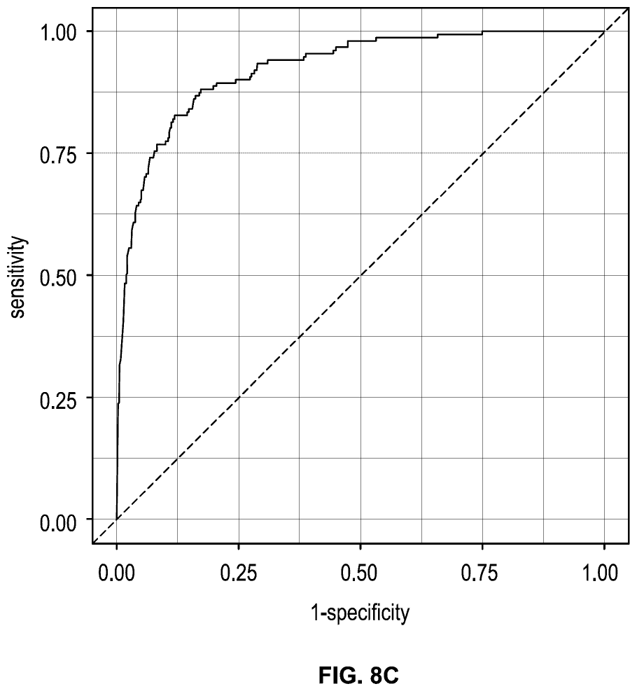

FIG. 8A illustrates the ROC curve for the determination of word groups related to cancer prognosis, plotted against a reference line indicating an equal rate of true positives and false positives. The area under this ROC curve is 0.9005. Table 5C below shows the value of various different statistical parameters of the selected random forest model for different probability thresholds. As the probability threshold increases (e.g., as the threshold for what is expected to be a determination that a given word group is associated with the predetermined topic becomes more restrictive), the sensitivity decreases while the specificity increases. Conversely, as the threshold probability decreases (e.g., as the threshold for what is expected to be a determination that a given word group is associated with the predetermined topic more inclusive), the sensitivity increases while the specificity decreases. The threshold probability that maximizes the sensitivity and specificity by the J-index is 0.5.

TABLE 5C

| Threshold | TN | FP | FN | TP | accuracy | sens | spec | ppv | npv | j_index |
|---|---|---|---|---|---|---|---|---|---|---|
| 0.5 | 3761 | 464 | 23 | 51 | 0.887 | 0.689 | 0.89 | 0.099 | 0.994 | 0.579 |
| 0.6 | 4053 | 172 | 33 | 41 | 0.952 | 0.554 | 0.959 | 0.192 | 0.992 | 0.513 |
| 0.7 | 4167 | 58 | 41 | 33 | 0.977 | 0.446 | 0.986 | 0.363 | 0.99 | 0.432 |
| 0.8 | 4206 | 19 | 58 | 16 | 0.982 | 0.216 | 0.996 | 0.457 | 0.986 | 0.212 |
| 0.9 | 4225 | 0 | 74 | 0 | 0.983 | 0 | 1 | NA | 0.983 | 0 |

However, the selection of the ideal probability threshold for the random forest model would also be informed by the purpose of the information. If the intent is to provide a list of the highest yield information but least inclusive information on cancer prognosis (e.g., containing few word groups that are irrelevant to cancer prognosis at the cost of missing all the word groups having to do with cancer prognosis), then a high probability threshold would be selected. If the intent is to provide a list of the most inclusive but less precise information on cancer prognosis (e.g., containing some word groups that may have less to do with cancer prognosis but capturing more word groups relevant to cancer prognosis), then a low probability threshold would be selected.

For each of the 42 consultations, the top ten word groups by probability of being related to cancer prognosis were used to predict the overall quality of risk communication across the entire consultation using an a priori defined hierarchy for quality assessment. This hierarchy designates quality across a spectrum of increasing granularity of communication: (0) not mentioned, (1) name only (without risk quantification), (2) generalization (“high”/“low”), (3) average percent incidence without timepoint, (4) average percent incidence with timepoint, (5) precision estimate accounting for patient-level characteristics. For each consultation, a reviewer manually analyzed the entire consultation and gave the consultation a score within the hierarchy, where the score generally indicated the highest-scoring word group of all word groups within that consultation. Similarly, a reviewer manually analyzed the top ten word groups identified by selected random forest model and gave the consultation a score within the hierarchy, where the score indicated the highest-scoring word group of the top ten highest-probability word groups. Table 5D below shows the comparison between the highest-scoring word group resulting from the manual review of the entire consultation and the highest-scoring word group resulting from the manual review of the top ten highest-probability word groups identified by the selected random forest model. Each cell indicates the number of consultations where the highest-scoring word group was the specific score indicated by the column and row headers of that cell. The ability of the selected random forest model to identify the overall quality of risk communication for cancer prognosis as measured by manual review across the entire consultation showed a 69% accuracy, a 63% sensitivity, and a 94% specificity.

TABLE 5D

Truth

| | | 0 | 1 | 2 | 3 | 4 | 5 |
|---|---|---|---|---|---|---|---|
| Prediction | 0 | 10 | 2 | 0 | 0 | 0 | 0 |
| | 1 | 0 | 1 | 0 | 1 | 0 | 0 |
| | 2 | 0 | 0 | 1 | 0 | 0 | 0 |
| | 3 | 0 | 0 | 0 | 0 | 0 | 0 |
| | 4 | 0 | 0 | 0 | 0 | 3 | 0 |
| | 5 | 0 | 0 | 0 | 1 | 0 | 0 |

FIG. 8B shows a linear regression plot of the scores given to various word groups for the “cancer prognosis” topic based on manual review, versus the probability that each respective word group is associated with cancer prognosis, with an overlaid trend line. As shown by the plot, the probability of being associated with cancer prognosis is well-correlated with the score given to the word group based on the manual review, indicating that the probability assigned by the model can be used to reliably score a consultation (e.g., grade the quality level of the consultation) with respect to cancer prognosis.

Once the random forest model was selected to identify word groups related to cancer prognosis, it was externally validated using a sample of 13 transcripts containing a total of 4,658 sentences. 151 of the sentences were related to cancer prognosis, and 4,507 sentences were not related to cancer prognosis. FIG. 8C illustrates the ROC curve for this validation, with an area under the curve of 0.925. The model's sensitivity and specificity were calculated for the prediction of word groups related to cancer prognosis at the probability threshold having the highest J-index (0.5) that was identified during training. The model's performance with the external validation dataset using this probability threshold is shown in Table 5E.

TABLE 5E

| | | | | | accu- | | | Balanced |
|---|---|---|---|---|---|---|---|---|
| Threshold | TN | FP | FN | TP | racy | sens | spec | Accuracy |
| 0.5 | 3811 | 696 | 24 | 127 | 0.845 | 0.841 | 0.846 | 0.843 |

For each consultation in the external validation dataset, the top ten word groups (e.g., sentences or phrases spoken by the healthcare provider) by probability of being related to cancer prognosis were used to predict the overall quality of risk communication across the entire consultation, using the same hierarchy described above. Table 5F below shows the comparison between the highest-scoring word group resulting from the manual review of the entire consultation and the highest-scoring word group resulting from the manual review of the top ten highest-probability word groups identified by the selected random forest model. Each cell indicates the number of consultations where the highest-scoring word group was the specific score indicated by the column and row headers of that cell. The ability of the selected random forest model to identify the overall quality of risk communication for urinary incontinence as measured by manual review across the entire consultation showed a 92.3% accuracy, a 95.8% sensitivity, and a 98.6% specificity.

TABLE 5F

Truth

| | | 0 | 1 | 2 | 3 | 4 | 5 |
|---|---|---|---|---|---|---|---|
| Prediction | 0 | 0 | 0 | 0 | 0 | 0 | 0 |
| | 1 | 0 | 0 | 0 | 0 | 0 | 0 |
| | 2 | 0 | 0 | 2 | 0 | 0 | 0 |
| | 3 | 0 | 0 | 0 | 1 | 1 | 0 |
| | 4 | 0 | 0 | 0 | 0 | 5 | 0 |
| | 5 | 0 | 0 | 0 | 0 | 0 | 4 |

As shown by these five examples, the quality of the top-ten highest probability sentences (or other word groups) for a given topic identified by the model for that topic is generally a good proxy for the quality of the entire consultation as a whole (which may include hundreds or even thousands of sentences). Identifying the top ten highest-probability sentences (or any other suitable number of the highest-probability sentences) using the models disclosed herein allows for a much more efficient analysis of the consultation, as only those identified sentences need to be reviewed and/or scored. For example, if a doctor wishes to review their consultation, the disclosed models can be used to identify the top ten highest-probability sentences, and the doctor can review only those sentences, instead of the entire consultation. In another example, the identified sentences can be sent to the patient afterward so that they may revisit the consultation and review what their doctor told them, without having to ready through the entire consultation. In a further example, large-scale reviews of doctor performance during consultations can be undertaken (e.g., by a hospital or healthcare system, by an insurance provider, etc.) more efficiently by reviewing only the sentences identified by the models, instead of the entirety of every consultation.

### Example 6

In the sixth example, validated NLP models (e.g., any of the models discussed herein) were used to extract sentences related to cancer prognosis (CP), life expectancy (LE), erectile dysfunction (ED), irritative urinary symptoms (IUS), and urinary incontinence (UI) from the 42 consultations.

These NLP models were trained on a corpus of 28,927 annotated sentences from other consultations. The models used Random Forest classifiers trained on 75% of the corpus, with 25% reserved for internal validation. They achieved strong overall performance for identifying topic-concordant sentences across domains, including CP, LE, ED, IUS, and UI, with AUROCs ranging from 0.84 to 0.99. In 20 independent consultations, the models selected the top 10 quality-graded sentences for each domain with topic-level accuracies ranging from 80% to 100%, when compared with manual coding.

To assess the effect of model confidence on summary content, ChatGPT4.0 was prompted to summarize each topic using NLP-identified text at increasing deciles of NLP-based probability thresholds for topic concordance. At each threshold, only sentences meeting or exceeding that probability were included in the input text. For each patient, ChatGPT summaries were created from both the raw consultation transcript (e.g., a probability threshold of 0%) and at NLP-extracted sentences for the 5 key topics at probability thresholds of 50%, 60%, 70%, 80% and 90% resulting in a total of 1,260 summaries (42 consultations, 5 topics per consultation, 6 different probability thresholds). The prompt instructed ChatGPT to: “Please summarize the following text on [topic] in the first-person context of an expert urological oncologist speaking to a patient.”

Each of the resulting 1,260 summaries was manually coded by at least two different coders for informational quality according to a 0-5 scoring hierarchy. The hierarchy used for summaries related to ED, UI, and IUS was: (0) not mentioned, (1) name only (without risk quantification), (2) generalization (“high”), (3) average percent incidence without timepoint, (4) average percent incidence with timepoint, (5) precision estimate accounting for patient-level characteristics. The hierarchy used for summaries related to LE was: (0) not mentioned, (2) generalization, (3) rough number of years, (4) probability of mortality/survival at a timepoint, and (5) specific number of years. The hierarchy used for summaries related to CP was: (0) not mentioned, (2) generalization, (3) probability of cancer mortality with no timeline, with treatment only, or without treatment only; (4) probability of cancer mortality at arbitrary timepoint with and without treatment; (5) probability of cancer mortality at the patient's life expectancy with and without treatment. Table 6A below shows the distribution of manually-coded quality scores across all 1,260 AI-generated summaries.

TABLE 6A

| | Overall | | CP | | ED | | UI | | IUS | | LE | |
|---|---|---|---|---|---|---|---|---|---|---|---|---|
| | N = 1,260 | | N = 252 | | N = 252 | | N = 252 | | N = 252 | | N = 252 | |
| 0 | 355 | (28%) | 30 | (12%) | 41 | (16%) | 134 | (53%) | 85 | (34%) | 65 | (26%) |
| 1 | 88 | (7.0%) | 4 | (1.6%) | 28 | (11%) | 12 | (4.8%) | 13 | (5.2%) | 31 | (12%) |
| 2 | 351 | (28%) | 109 | (43%) | 65 | (26%) | 105 | (42%) | 31 | (12%) | 41 | (16%) |
| 3 | 190 | (15%) | 82 | (33%) | 42 | (17%) | 1 | (0.4%) | 39 | (15%) | 26 | (10%) |
| 4 | 194 | (15%) | 27 | (11%) | 53 | (21%) | 0 | (0%) | 28 | (11%) | 86 | (34%) |
| 5 | 82 | (6.5%) | 0 | (0%) | 23 | (9.1%) | 0 | (0%) | 56 | (22%) | 3 | (1.2%) |

Table 6B below shows the distribution of manually-coded quality scores for each probability threshold.

TABLE 6B

| | | | | | |
|---|---|---|---|---|---|
| Overall | CP | ED | UI | IUS | LE |
| N = 210 | N = 42 | N = 42 | N = 42 | N = 42 | N = 42 |

Probability Threshold: 0 (full consultation)

| | | | | | | | | | | | | |
|---|---|---|---|---|---|---|---|---|---|---|---|---|
| 0 | 55 | (26%) | 0 | (0%) | 9 | (21%) | 10 | (24%) | 21 | (50%) | 15 | (36%) |
| 1 | 23 | (11%) | 2 | (5%) | 6 | (14%) | 8 | (19%) | 4 | (10%) | 3 | (7%) |
| 2 | 73 | (35%) | 22 | (52%) | 10 | (24%) | 13 | (31%) | 17 | (40%) | 11 | (26%) |
| 3 | 28 | (13%) | 14 | (33%) | 7 | (17%) | 3 | (7%) | 0 | (0%) | 4 | (10%) |
| 4 | 21 | (10%) | 4 | (10%) | 7 | (17%) | 8 | (19%) | 0 | (0%) | 2 | (5%) |
| 5 | 10 | (5%) | 0 | (0%) | 3 | (7%) | 0 | (0%) | 0 | (0%) | 7 | (17%) |

Probability Threshold: 0.5

| | | | | | | | | | | | | |
|---|---|---|---|---|---|---|---|---|---|---|---|---|
| 0 | 45 | (21%) | 1 | (2%) | 6 | (14%) | 10 | (24%) | 19 | (45%) | 9 | (21%) |
| 1 | 15 | (7%) | 0 | (0%) | 5 | (12%) | 4 | (10%) | 1 | (2%) | 5 | (12%) |
| 2 | 65 | (31%) | 23 | (55%) | 10 | (24%) | 7 | (17%) | 21 | (50%) | 4 | (10%) |
| 3 | 38 | (18%) | 14 | (33%) | 7 | (17%) | 6 | (14%) | 0 | (0%) | 10 | (24%) |
| 4 | 33 | (16%) | 4 | (10%) | 10 | (24%) | 15 | (36%) | 0 | (0%) | 4 | (5%) |
| 5 | 14 | (7%) | 0 | (0%) | 4 | (10%) | 0 | (0%) | 0 | (0%) | 10 | (24%) |

Probability Threshold: 0.6

| | | | | | | | | | | | | |
|---|---|---|---|---|---|---|---|---|---|---|---|---|
| 0 | 43 | (20%) | 2 | (5%) | 5 | (12%) | 9 | (21%) | 19 | (45%) | 8 | (19%) |
| 1 | 16 | (7%) | 0 | (0%) | 5 | (12%) | 5 | (12%) | 2 | (5%) | 4 | (10%) |
| 2 | 65 | (31%) | 22 | (52%) | 11 | (26%) | 7 | (17%) | 21 | (50%) | 4 | (10%) |
| 3 | 35 | (17%) | 13 | (31%) | 7 | (17%) | 5 | (12%) | 0 | (0%) | 10 | (24%) |
| 4 | 38 | (18%) | 5 | (12%) | 12 | (29%) | 16 | (38%) | 0 | (0%) | 5 | (12%) |
| 5 | 13 | (6%) | 0 | (0%) | 2 | (5%) | 0 | (0%) | 0 | (0%) | 11 | (26%) |

Probability Threshold: 0.7

| | | | | | | | | | | | | |
|---|---|---|---|---|---|---|---|---|---|---|---|---|
| 0 | 48 | (23%) | 2 | (5%) | 5 | (12%) | 10 | (24%) | 21 | (50%) | 10 | (24%) |
| 1 | 14 | (7%) | 1 | (2%) | 5 | (12%) | 5 | (12%) | 2 | (5%) | 1 | (2%) |
| 2 | 59 | (28%) | 19 | (45%) | 11 | (26%) | 5 | (12%) | 19 | (45%) | 5 | (12%) |
| 3 | 34 | (16%) | 14 | (33%) | 8 | (19%) | 5 | (12%) | 0 | (0%) | 7 | (17%) |
| 4 | 37 | (18%) | 6 | (14%) | 8 | (19%) | 16 | (38%) | 0 | (0%) | 7 | (17%) |
| 5 | 18 | (9%) | 0 | (0%) | 5 | (12%) | 1 | (2%) | 0 | (0%) | 12 | (29%) |

Probability Threshold: 0.8

| | | | | | | | | | | | | |
|---|---|---|---|---|---|---|---|---|---|---|---|---|
| 0 | 59 | (28%) | 7 | (17%) | 6 | (14%) | 11 | (26%) | 22 | (52%) | 13 | (31%) |
| 1 | 11 | (5%) | 1 | (2%) | 5 | (12%) | 3 | (7%) | 2 | (5%) | 0 | (0%) |
| 2 | 54 | (26%) | 13 | (31%) | 11 | (26%) | 6 | (14%) | 18 | (43%) | 6 | (14%) |
| 3 | 31 | (15%) | 14 | (33%) | 7 | (17%) | 4 | (10%) | 0 | (0%) | 6 | (14%) |
| 4 | 38 | (18%) | 7 | (17%) | 8 | (19%) | 17 | (40%) | 0 | (0%) | 6 | (14%) |
| 5 | 17 | (8%) | 0 | (0%) | 5 | (12%) | 1 | (2%) | 0 | (0%) | 11 | (26%) |

Probability Threshold: 0.9

| | | | | | | | | | | | | |
|---|---|---|---|---|---|---|---|---|---|---|---|---|
| 0 | 105 | (50%) | 18 | (43%) | 10 | (24%) | 15 | (36%) | 32 | (76%) | 30 | (71%) |
| 1 | 9 | (4%) | 0 | (0%) | 2 | (5%) | 6 | (14%) | 1 | (2%) | 0 | (0%) |
| 2 | 35 | (17%) | 10 | (24%) | 12 | (29%) | 3 | (7%) | 9 | (21%) | 1 | (2%) |
| 3 | 24 | (11%) | 13 | (31%) | 6 | (14%) | 3 | (7%) | 0 | (0%) | 2 | (5%) |
| 4 | 27 | (13%) | 1 | (2%) | 8 | (19%) | 14 | (33%) | 0 | (0%) | 4 | (10%) |
| 5 | 10 | (5%) | 0 | (0%) | 4 | (10%) | 1 | (2%) | 0 | (0%) | 5 | (12%) |

To evaluate the quality of AI-generated summaries, several metrics were employed, including the proportion of sentences specifically relating to the domain of interest, quality score, probability of a quality score ≤3, and probability of topic concordance. To assess the impact of NLP probability thresholds on these outcomes, generalized linear mixed-effects models (GLMMs) with both linear and logit link functions were utilized. Each model was adjusted for the coder and included an interaction term between the NLP probability threshold and domain. A random intercept was included to account for clustering by consultation ID. Model diagnostics were performed to verify the assumptions of the GLMMs, including checks for normality of residuals, homoscedasticity, and multicollinearity.

As a sensitivity analysis, it was tested whether improvements in ChatGPT performance were due to score inflation beyond the quality of physician communication. The consultations themselves were manually coded according to the same hierarchies, and for each topic in each consultation, the difference between the ChatGPT quality score (the manually-coded scores of the ChatGPT-generated summaries) and the highest manual score assigned by the coders for that topic was determined, such that scores were standardized for quality of physician communication as manually coded in the consultation transcript. The highest score achievable was a zero, or no difference from the quality of physician communication in manual coding. These capped delta scores were analyzed using a linear mixed-effects model to assess whether increasing probability thresholds led to summaries that more closely aligned with the best available physician communication. An exploratory analyses stratified by patient race was also performed to assess whether improvements in AI summary quality differed across subgroups. All statistical analyses were conducted using R statistical software. Two-sided tests were employed, with a significance level set at 0.05.

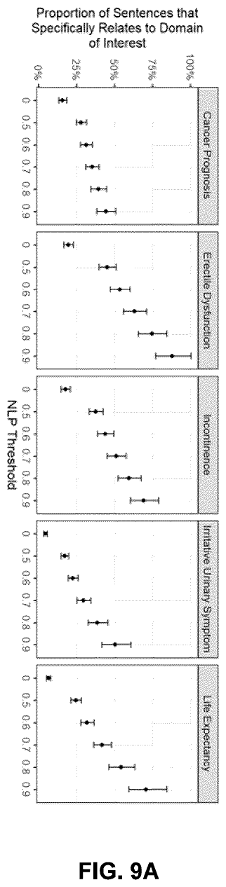

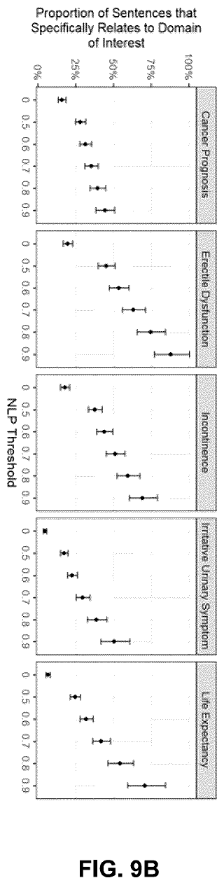

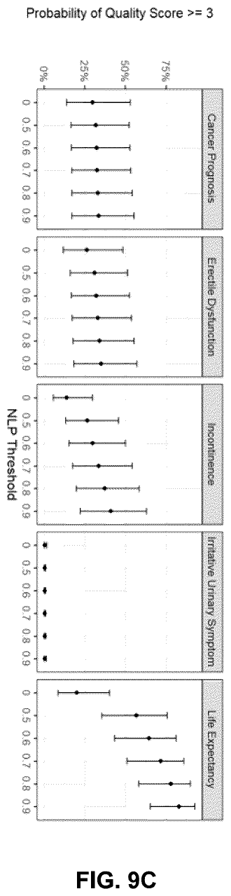

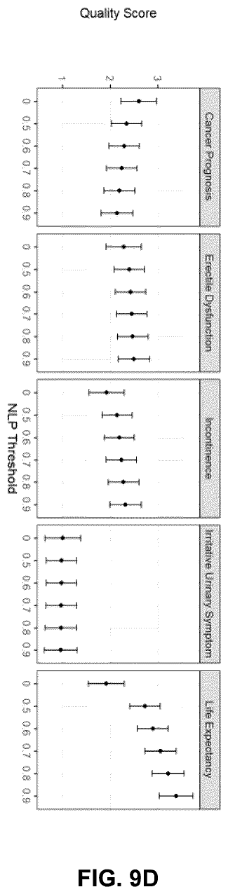

Referring to FIG. 9A, for each of the five topics, the proportion of sentences related to the domain of interest increased with the NLP probability threshold, though at different rates. For each 10% increase in the NLP probability threshold for CP, the proportion of topic-related sentences increased by: 12% for CP (IRR 1.12, 95% CI 1.10-1.15, p<0.001); 18% for ED (IRR 1.18, 95% CI 1.16-1.20, p<0.001); 16% for UI (IRR 1.16, 95% CI 1.14-1.19, p<0.001); 31% for UIS (IRR 1.31, 95% CI 1.26-1.36, p<0.001); and 30% for LE (IRR 1.30, 95% CI 1.26-1.35, p<0.001).

Referring to FIG. 9B, the probability of overall topic concordance increased with higher NLP probability thresholds across different topics. Compared with summaries generated from raw transcripts, a 60% threshold increased the odds of topic concordance by 2.66-fold for CP (OR 2.66, 95% CI 1.99-3.54, p<0.001); 2.53-fold for ED (OR 2.53, 95% CI 2.10-3.05, p<0.001); 2.21-fold for UI (OR 2.21, 95% CI 1.78-2.74, p<0.001); 1.80-fold for UIS (OR 1.80, 95% CI 1.44-2.26, p<0.001); and 2.08-fold for LE (OR 2.08, 95% CI 1.72-2.52, p<0.001).

Referring to FIG. 9C, the probability of quality score greater than or equal 3 as inclusion of a quantified estimate of risk, increased with higher NLP probability threshold across different topics. There was a significant increase in the odds of a quantified estimate of risk being provided by 1.18-fold for UI (OR 1.18, 95% CI 1.08 to 1.28, p<0.001) and 1.39-fold for LE (OR 1.39, 95% CI 1.26 to 1.54, p<0.001) for each 10% increase in NLP threshold. For CP, ED, and UIS, the odds of a quantified estimate or risk were not statistically different with increasing NLP threshold.

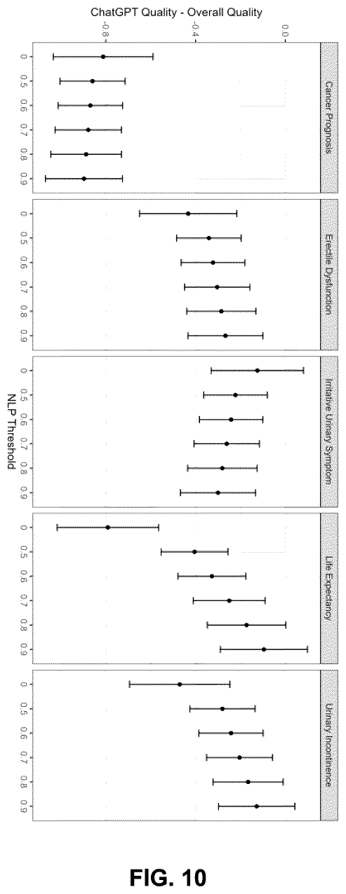

Referring to FIG. 9D, the quality score of AI-generated summaries varied with changes in the NLP probability threshold across different topics, though clinically meaningful change was only observed for life expectancy (LE). For LE, the quality score increased by 0.16 points (+0.16, 95% CI 0.12 to 0.20, p<0.001) for each 10% increase in the NLP probability threshold, corresponding to a clinically meaningful change (1 point) at an NLP threshold of 60% compared with the raw transcript. For UI, the quality score increased by 0.04 points (+0.04, 95% CI 0.01 to 0.08, p=0.011), and for CP, it decreased by 0.05 points (−0.05, 95% CI −0.09 to −0.02, p=0.004). However, referring to FIG. 10, these changes were not clinically meaningful at any NLP threshold compared with the raw transcript. For ED and UIS, there was not a statistically significant change in the quality score. In the sensitivity analysis where quality scores were capped at the highest manual score per topic, LE remained the only topic that exhibited statistically and clinically meaningful improvement in quality score, as shown in FIG. 10.

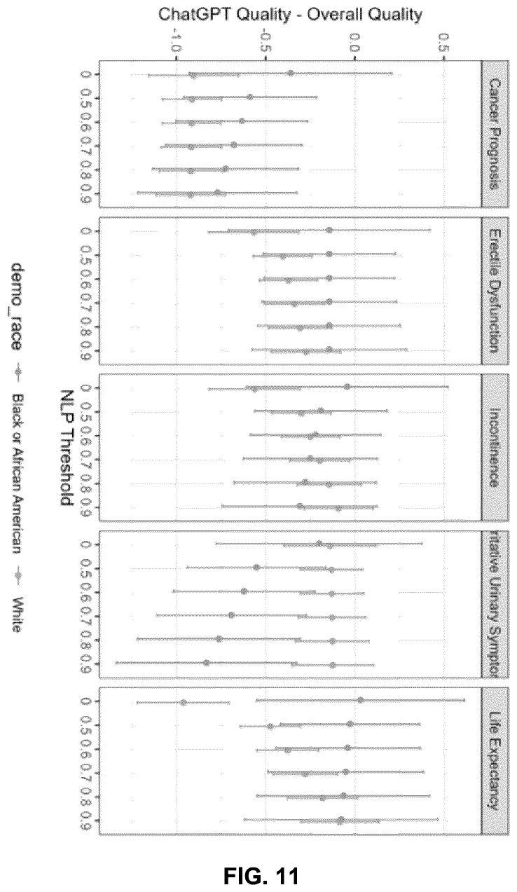

Referring to FIG. 11, in a subgroup analysis stratified by race, Black patients demonstrated lower improvements in summary quality compared with White patients for LE (estimate 0.11, p=0.028) and for UI (estimate 0.08, p=0.050). No significant racial differences were observed for CP, ED, or UIS.

While ChatGPT-4.0 can generate summaries of key data from treatment consultations, these summaries may lack clarity or omit essential details when created directly from raw consultation transcripts. The potential of integrating advanced NLP techniques to enhance the quality of these summaries is appealing but needs to be systematically evaluated. To address this, combining NLP pre-processing to identify topic-specific content prior to summarization by ChatGPT was experimented with, and topic concordance and the quality of risk information in ChatGPT-generated summaries of prostate cancer consultations was assessed. The findings demonstrate that integrating NLP preprocessing with ChatGPT, compared to using raw transcripts alone (i.e., 0% threshold), significantly improves topic concordance in summaries of prostate cancer consultations. Specifically, compared to raw transcript summaries (0% threshold), increasing the NLP probability threshold led to notable improvements in topic concordance. For each 10% increase in threshold, the proportion of topic-related sentences increased by over 30% for IUS and LE, while the odds of ChatGPT summaries being topic-concordant increased by more than 50% for IUS, CP, and ED. However, quality of risk communication and the presence of quantified risk estimates varied by topic, with notable improvement observed only for LE and UI summaries.

Patients with prostate cancer must navigate complex tradeoffs in treatment decisions, including balancing CP with side effects such as urinary and sexual dysfunction or differences in LE across different treatment options. However, when faced with the difficult conversation about their disease, patients are often overwhelmed by the amount of information presented, making it challenging to process and retain critical details. Given the complexity of these conversations, physicians may not always present information in a way that is tailored to the patient's needs. Since informed decision-making relies on a clear understanding of treatment risks and benefits, improving the quality and personalization of consultation summaries, such as those generated by AI, may be helpful in facilitating shared decision-making. AI summaries can help patients revisit key tradeoffs, recall specific risk information, and ultimately help them make more informed decisions. The data shows that NLP preprocessing significantly increased relevant content in AI-generated summaries, improving topic concordance and ensuring summaries more precisely communicated risk for select topics. This improvement may be important in helping patients distill the large amount of information from a consultation down to the essential aspects of prostate cancer decision-making.

Generally, any of the methods disclosed herein can be implemented using a system having a control system with one or more processors, and a memory device storing machine-readable instructions. The control system can be coupled to the memory device, and methods can be implemented when the machine-readable instructions are executed by at least one of the processors of the control system. The methods can also be implemented using a computer program product (such as a non-transitory computer readable medium) comprising instructions that when executed by a computer, cause the computer to carry out the steps of the methods.

One or more elements or aspects or steps, or any portion(s) thereof, from one or more of any of claims or Alternative Implementations below can be combined with one or more elements or aspects or steps, or any portion(s) thereof, from one or more of any of the other claims or Alternative Implementations or combinations thereof, to form one or more additional implementations and/or claims of the present disclosure.

### Alternative Implementations

Alternative Implementation 1. A method of reviewing a consultation between a healthcare provider and a patient, the method comprising: receiving data associated with a transcript of the consultation between the healthcare provider and the patient; analyzing the data to extract a plurality of word groups, each word group including or more words spoken by the healthcare provider during the consultation; and determining whether each respective word group of the plurality of word groups is associated with a predetermined topic.

Alternative Implementation 2. The method of Alternative Implementation 1, wherein determining whether each respective word group is associated with the predetermined topic includes: selecting a probability threshold; determining, for each respective word group, a probability that the respective word group is associated with the predetermined topic; classifying any word group of the plurality of word groups that satisfies the probability threshold as being associated with the predetermined topic; and classifying any word group of the plurality of word groups that does not satisfy the probability threshold as not being associated with the predetermined topic.

Alternative Implementation 3. The method of Alternative Implementation 1 or Alternative Implementation 2, wherein determining whether each respective word group is associated with the predetermined topic includes determining, for each respective word group, a probability that the respective word group is associated with the predetermined topic, the probability being greater than a threshold probability indicating that the respective word group is associated with the predetermined topic.

Alternative Implementation 4. The method of any one of Alternative Implementations 1 to 3, wherein the determination of whether each respective word group is associated with the predetermined topic is based on a plurality of distinct estimates of whether the respective word group is associated with the predetermined topic.

Alternative Implementation 5. The method of any one of Alternative Implementations 1 to 4, wherein determining whether each respective word group is associated with the predetermined topic includes: generating a plurality of distinct estimates of whether the respective word group is associated with the topic, each distinct estimate indicating that the respective word group is associated with the predetermined topic or not associated with the predetermined topic; and determining a probability that the respective word group is associated with the predetermined topic based at least in part on the plurality of distinct estimates for the respective word group.

Alternative Implementation 6. The method of Alternative Implementation 5, wherein the probability for each respective word group is a percentage of the distinct estimates for the respective word group that indicate that the respective word group is associated with the predetermined topic.

Alternative Implementation 7. The method of Alternative Implementation 5 or Alternative Implementation 6, wherein the plurality of distinct estimates is generated by a random forests model, the random forests model including a plurality of decision trees that are each configured generate a respective one of the plurality of distinct estimates.

Alternative Implementation 8. The method of Alternative Implementation 7, wherein each of the plurality of decision trees is configured to determine whether each respective word group includes one or more tokens.

Alternative Implementation 9. The method of Alternative Implementation 8, wherein each of the plurality of decision trees is configured to determine whether each respective word group includes each token of a random subset of the one or more tokens.

Alternative Implementation 10. The method of any one of Alternative Implementations 1 to 9, wherein determining whether each respective word group is associated with the predetermined topic is based on a determination, for each respective word group, of whether the respective word group includes one or more tokens.

Alternative Implementation 11. The method of Alternative Implementation 9 or Alternative Implementation 10, wherein each of the one or more tokens is a word, a phrase containing a plurality of words, a word stem, or a word root.

Alternative Implementation 12. The method of any one of Alternative Implementations 9-11, wherein each of the one or more tokens is associated with the predetermined topic.

Alternative Implementation 13. The method of any one of Alternative Implementations 1 to 12, wherein each of the plurality of word groups includes at least one word.

Alternative Implementation 14. The method of any one of Alternative Implementations 1 to 13, wherein each of the plurality of word groups is a word, a phrase including a plurality of words, or a sentence containing a plurality of words.

Alternative Implementation 15. The method of any one of Alternative Implementations 1 to 16, wherein each of the plurality of word groups includes a plurality of letters.

Alternative Implementation 17. The method of any one of Alternative Implementations 1 to 18, wherein the data associated with the communication between the healthcare provider and the patient includes text data, audio data, or both.

Alternative Implementation 18. The method of any one of Alternative Implementations 1 to 19, wherein the predetermined topic is a medical condition, a life expectancy following diagnosis of the medical condition, a result of a treatment of the medical condition, a side effect of the medical condition, a side effect of the treatment of the medical condition, or any combination thereof.

Alternative Implementation 19. The method of Alternative Implementation 18, wherein the medical condition is prostate cancer.

Alternative Implementation 20. The method of Alternative Implementation 18 or Alternative Implementation 19, wherein the side effect of the medical condition and/or the side effect of the treatment of the medical condition includes erectile dysfunction, urinary incontinence, irritative lower urinary tract symptoms, or any combination thereof.

Alternative Implementation 21. The method of any one of Alternative Implementations 1 to 21, further comprising assigning a score to at least one word group of the plurality of word groups that is determined to be associated with the predetermined topic, the score indicating a level of detail of the at least one word group.

Alternative Implementation 22. A method of generating a random forest model to score a consultation between a healthcare provider and a patient, the method comprising: receiving data associated with one or more consultations between the healthcare provider and the patient; analyzing the data to extract a plurality of word groups; forming a training dataset from a first portion of the plurality of word groups, the training dataset including a (i) plurality of training word groups and (ii) for each respective training word group, an indication of whether the respective training word group is associated with a predetermined topic; generating a plurality of random forest models based on the plurality of training word groups, each of the plurality of random forest models being configured to determine if each of the plurality of training word groups is associated with the predetermined topic; and selecting one of the plurality of random forest models, the selected random forest model having a highest accuracy in determining whether each of the plurality of training word groups is associated with the predetermined topic among all of the plurality of random forest models.

Alternative Implementation 23. The method of Alternative Implementation 22, wherein each respective random forest model includes a plurality of decision trees, each decision tree of each respective random forest model being configured to estimate whether each respective training word group is associated with the predetermined topic.

Alternative Implementation 24. The method of Alternative Implementation 23, wherein each respective random forest model is configured to determine a probability that each respective training word group is associated with the predetermined topic, the determination by each respective random forest model being based at least in part on the estimate of each of the plurality of the decision trees of the respective random forest model.

Alternative Implementation 25. The method of Alternative Implementation 24, wherein the probability that each respective training word group is associated with the predetermined topic is a percentage of the plurality of decision trees of the respective random forest model that estimate that the respective training word group is associated with the predetermined topic.

Alternative Implementation 26. The method of Alternative Implementation 25, wherein each respective decision tree of each respective random forest model includes a plurality of nodes, each node determining whether each respective more training word group includes a corresponding token.

Alternative Implementation 27. The method of any one of Alternative Implementation 22 to 26, wherein each respective one of the plurality of random forest models includes a distinct combination of (i) a number of one or more decision trees and (ii) a number of tokens searched for by the one or more decision trees of the respective random forest model.

Alternative Implementation 28. The method of Alternative Implementation 27, wherein the number of one or more decision trees in each respective random forest model is greater than or equal to 100, and less than or equal to 10,000.

Alternative Implementation 29. The method of Alternative Implementation 27 or Alternative Implementation 28, wherein the number of tokens searched by the one or more decision trees of each respective random forest model is greater than or equal to 500, and less than or equal to 10,000.

Alternative Implementation 30. The method of any one of Alternative Implementations 22 to 29, further comprising: forming a validation dataset from a second portion of the plurality of word groups, the validation dataset including a (i) plurality of validation word groups and (ii) for each respective validation word group, an indication of whether the respective validation word group is associated with a predetermined topic; and validating the selected one of the plurality of random forests models using the validation dataset.

Alternative Implementation 31. A method of reviewing a consultation between a healthcare provider and a patient, the method comprising: receiving data associated with the consultation, the data including audio data reproducible as audio of the consultation, video data reproducible as a video of the consultation, or both; extracting a transcript of the consultation from the received data; identifying a plurality of word groups within the transcript, each word group including one or more words spoken by the healthcare provider during the consultation; determining a probability that each respective word group is associated with a predetermined topic based on which of a plurality of predetermined tokens are identified in the respective word group; and determining an overall score for the consultation based at least in part on the determined probability for at least one of the plurality of word groups.

Alternative Implementation 32. The method of Alternative Implementation 31, wherein each of the identified word groups is a sentence.

Alternative Implementation 33. The method of Alternative Implementation 31 or Alternative Implementation 32, further comprising assigning a score to one or more respective word groups of the plurality of word groups based on (i) the probability of the respective word group being associated with the topic, (ii) the tokens of the plurality of predetermined tokens identified in the respective word group, or (iii) both (i) and (ii).

Alternative Implementation 34. The method of Alternative Implementation 33, wherein the one or more respective word groups to which the score was assigned includes each of the plurality of word groups having at least a threshold probability of being associated with the predetermined topic

Alternative Implementation 35. The method of Alternative Implementation 33, wherein the one or more respective word groups to which the score was assigned includes a set of n word groups having a highest probability of being associated with the predetermined topic among all of the plurality of word groups.

Alternative Implementation 36. The method of any one of Alternative Implementations 33 to 35, wherein determining the overall score for the consultation includes determining an average score among the one or more respective word groups to which the score was assigned.

Alternative Implementation 37. The method of any one of Alternative Implementations 33 to 35, wherein determining the overall score for the consultation includes determining a weighted average score among the one or more respective word groups to which the score was assigned.

Alternative Implementation 38. The method of Alternative Implementation 37, wherein each of the one or more respective word groups to which the score was assigned is weighted based on a location of the respective word group within the transcript of the consultation.

Alternative Implementation 39. The method of Alternative Implementation 37, wherein each of the one or more respective word groups to which the score was assigned is weighted based a length of the respective word group relative to a high threshold length and a low threshold length.

Alternative Implementation 40. The method of Alternative Implementation 39, wherein each respective word group is a sentence and the length of the respective word group is a number of words within the sentence, and wherein the high threshold length and the low threshold length are each a specific number of words.

Alternative Implementation 41. The method of any one of Alternative Implementations 31 to 40, further comprising: generating a message containing at least the overall score for the consultation; and transmitting the message to the healthcare provider.

Alternative Implementation 42. The method of Alternative Implementation 41, wherein generating and transmitting the message are performed automatically in response to determining the overall score for the consultation.

Alternative Implementation 43. The method of Alternative Implementation 41 or Alternative Implementation 42, wherein the message further contains (i) each word group to which a score was assigned, (ii) each respective word group having at least the threshold probability of being associated with the predetermined topic, or (iii) each of the set of n word groups having a highest probability of being associated with the predetermined topic among all of the plurality of word groups.

Alternative Implementation 44. The method of Alternative Implementation 43, wherein the message further contains, for each respective word group included in message, the probability that the respective word group is associated with the predetermined topic.

Alternative Implementation 45. A system comprising a control system configured to implement the method of any one of Alternative Implementations 1 to 44.

Alternative Implementation 46. A computer program product comprising instructions which, when executed by a computer, cause the computer to carry out the method of any one of Alternative Implementations 1 to 44.

Alternative Implementation 47. The computer program product of Alternative Implementation 46, wherein the computer program product is a non-transitory computer readable medium.

Alternative Implementation 48. A system comprising: a memory device having stored thereon machine-readable instructions; and a control system including one or more processors configured to execute the machine-readable instructions to: receive data associated with a transcript of the consultation between the healthcare provider and the patient; analyze the data to extract a plurality of word groups, each word group including or more words spoken by the healthcare provider during the consultation; determine whether each respective word group of the plurality of word groups is associated with a predetermined topic.

Alternative Implementation 49. A system comprising: a memory device having stored thereon machine-readable instructions; and a control system including one or more processors configured to execute the machine-readable instructions to: receive data associated with one or more consultations between the healthcare provider and the patient; analyze the data to extract a plurality of word groups; form a training dataset from a first portion of the plurality of word groups, the training dataset including a (i) plurality of training word groups and (ii) for each respective training word group, an indication of whether the respective training word group is associated with a predetermined topic; generate a plurality of random forest models based on the plurality of training word groups, each of the plurality of random forest models being configured to determine if each of the plurality of training word groups is associated with the predetermined topic; and select one of the plurality of random forest models, the selected random forest model having a highest accuracy in determining whether each of the plurality of training word groups is associated with the predetermined topic among all of the plurality of random forest models.

Alternative Implementation 50. A system comprising: a memory device having stored thereon machine-readable instructions; and a control system including one or more processors configured to execute the machine-readable instructions to: receive data associated with the consultation, the data including audio data reproducible as audio of the consultation, video data reproducible as a video of the consultation, or both; extract a transcript of the consultation from the received data; identify a plurality of word groups within the transcript, each word group including one or more words spoken by the healthcare provider during the consultation; determine whether each respective word group is associated with a predetermined topic based on which of a plurality of predetermined tokens are identified in the respective word group; determine a probability that each respective word group is associated with a predetermined topic based on which of a plurality of predetermined tokens are identified in the respective word group; and determine an overall score for the consultation based at least in part on the determined probability for at least one of the plurality of word groups.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations or alternative implementations according to aspects of the present disclosure may combine any number of features from any of the implementations described herein, such as, for example, in the alternative implementations described above.

What is claimed is:

1. A method of reviewing a consultation between a healthcare provider and a patient, the method comprising:
receiving data associated with a transcript of the consultation between the healthcare provider and the patient;
analyzing the data to extract a plurality of word groups, each word group including or more words spoken by the healthcare provider during the consultation; and
determining whether each respective word group of the plurality of word groups is associated with a predetermined topic.

2. The method of claim 1, wherein determining whether each respective word group is associated with the predetermined topic includes:
selecting a probability threshold;
determining, for each respective word group, a probability that the respective word group is associated with the predetermined topic;
classifying any word group of the plurality of word groups that satisfies the probability threshold as being associated with the predetermined topic; and
classifying any word group of the plurality of word groups that does not satisfy the probability threshold as not being associated with the predetermined topic.

3. The method of claim 1, wherein determining whether each respective word group is associated with the predetermined topic includes:
generating a plurality of distinct estimates of whether the respective word group is associated with the topic, each distinct estimate indicating that the respective word group is associated with the predetermined topic or not associated with the predetermined topic; and
determining a probability that the respective word group is associated with the predetermined topic based at least in part on the plurality of distinct estimates for the respective word group.

4. The method of claim 3, wherein the probability for each respective word group is a percentage of the distinct estimates for the respective word group that indicate that the respective word group is associated with the predetermined topic.

5. The method of claim 3, wherein the plurality of distinct estimates is generated by a random forests model, the random forests model including a plurality of decision trees that are each configured generate a respective one of the plurality of distinct estimates.

6. The method of claim 1, wherein determining whether each respective word group is associated with the predetermined topic is based on a determination, for each respective word group, of whether the respective word group includes one or more tokens.

7. The method of claim 6, wherein each of the one or more tokens is a word, a phrase containing a plurality of words, a word stem, or a word root.

8. The method of claim 1, wherein each of the plurality of word groups is a word, a phrase including a plurality of words, or a sentence containing a plurality of words.

9. The method of claim 1, wherein the data associated with the communication between the healthcare provider and the patient includes text data, audio data, or both.

10. The method of claim 1, wherein the predetermined topic is a medical condition, a life expectancy following diagnosis of the medical condition, a result of a treatment of the medical condition, a side effect of the medical condition, a side effect of the treatment of the medical condition, or any combination thereof.

11. The method of claim 1, further comprising assigning a score to at least one word group of the plurality of word groups that is determined to be associated with the predetermined topic, the score indicating a level of detail of the at least one word group.

12. A method of reviewing a consultation between a healthcare provider and a patient, the method comprising:
receiving data associated with the consultation, the data including audio data reproducible as audio of the consultation, video data reproducible as a video of the consultation, or both;
extracting a transcript of the consultation from the received data;
identifying a plurality of word groups within the transcript, each word group including one or more words spoken by the healthcare provider during the consultation;
determining a probability that each respective word group is associated with a predetermined topic based on which of a plurality of predetermined tokens are identified in the respective word group; and
determining an overall score for the consultation based at least in part on the determined probability for at least one of the plurality of word groups.

13. The method of claim 12, wherein each of the identified word groups is a sentence.

14. The method of claim 12, further comprising assigning a score to one or more respective word groups of the plurality of word groups based on (i) the probability of the respective word group being associated with the topic, (ii) the tokens of the plurality of predetermined tokens identified in the respective word group, or (iii) both (i) and (ii).

15. The method of claim 14, wherein the one or more respective word groups to which the score was assigned includes (i) each of the plurality of word groups having at least a threshold probability of being associated with the predetermined topic or (ii) a set of n word groups having a highest probability of being associated with the predetermined topic among all of the plurality of word groups.

16. The method of claim 14, wherein determining the overall score for the consultation includes determining an average score among the one or more respective word groups to which the score was assigned or a weighted average score among the one or more respective word groups to which the score was assigned.

17. The method of claim 16, wherein each of the one or more respective word groups to which the score was assigned is weighted based on (i) a location of the respective word group within the transcript of the consultation, (ii) a length of the respective word group relative to a high threshold length and a low threshold length, or (iii) both (i) and (ii).

18. The method of claim 17, wherein each respective word group is a sentence and the length of the respective word group is a number of words within the sentence, and wherein the high threshold length and the low threshold length are each a specific number of words.

19. The method of claim 12, further comprising, in response to determining the overall score for the consultation, automatically generating a message containing at least the overall score for the consultation and transmitting the message to the healthcare provider.

20. The method of claim 19, wherein the message further contains (i) each word group to which a score was assigned, (ii) each respective word group having at least the threshold probability of being associated with the predetermined topic, or (iii) each of the set of n word groups having a highest probability of being associated with the predetermined topic among all of the plurality of word groups, and wherein the message further contains, for each respective word group included in message, the probability that the respective word group is associated with the predetermined topic.
